(19) United States
(12) Patent Application Publication
(10) Pub. No.: US 2026/0272716 A1
(43) Pub. Date: Sep. 17, 2026

(54) GOGGLE HAVING INTEGRATED FILM CANISTERS

(71) Applicant: 100% Speedlab, LLC, San Diego, CA (US)

(72) Inventors: Ludovic Francis BOINNARD, San Diego, CA (US); Marc Guy BLANCHARD, Solana Beach, CA (US); Shaun RYAN, San Diego, CA (US); Andrew HURSH, San Diego, CA (US); Gautier PAULIN, Uzes (FR)

(21) Appl. No.: 19/445,013

(22) Filed: Jan. 9, 2026

Related U.S. Application Data
(60) Provisional application No. 63/770,195, filed on Mar. 11, 2025.

(51) Int. Cl. A61F 9/02; A63B 71/10

(52) U.S. Cl. CPC A61F 9/025; A61F 9/029; A63B 71/10

(57) ABSTRACT

A protective goggle in accordance with the present teachings may include a pair of film canisters integrated into a goggle frame of the goggle. At least a portion of each of the film canisters is formed by an integral part of the goggle frame. The pair of canisters are configured to facilitate indexing a protective lens film across a front surface of a lens of the goggle. The lens of the goggle is configured to be operatively coupled to a front side of the goggle frame utilizing at least one latch mechanism of the goggle frame.

### CROSS-REFERENCES

The following applications and materials are incorporated herein by reference, in their entireties, for all purposes: U.S. Provisional Patent Application Ser. No. 63/770,195, filed Mar. 11, 2025 and U.S. Nonprovisional patent application Ser. No. 16/824,525, Filed Mar. 19, 2020.

### FIELD

This disclosure relates to systems and methods for protective eyewear. More specifically, the disclosed embodiments relate to goggles used in sports such as motocross and skiing.

### INTRODUCTION

Protective sports goggles are worn by users for various sports or activities, such as motorsports, powersports, snow sports, watersports, biking, or the like, to protect the user's eyes. Such protective sports goggles may become covered in dirt, snow, and/or other debris when worn by a user, which may obstruct the user's view when performing outdoor sports or activities.

### SUMMARY

The present disclosure provides systems, apparatuses, and methods relating to protective goggles.

In some examples, a protective goggle includes: a goggle frame including a first frame segment forming a lens receiving area configured to receive a lens of the goggle, wherein the goggle frame further comprises: a first integral canister configured to contain a supply roll of a lens film; and a second integral canister configured to contain a take-up roll of the lens film; wherein a first integral part of the goggle frame forms at least a first portion of the first integral canister and a second integral part of the goggle frame forms at least a second portion of the second integral canister.

Features, functions, and advantages may be achieved independently in various embodiments of the present disclosure, or may be combined in yet other embodiments, further details of which can be seen with reference to the following description and drawings.

### BRIEF DESCRIPTION OF THE DRAWINGS

FIG. 1 is a schematic diagram of an illustrative goggle in accordance with aspects of the present disclosure.

FIG. 2 is a front view of an illustrative example of the goggle of FIG. 1.

FIG. 3 is a rear view of the goggle of FIG. 2.

FIG. 4 is a top view of the goggle of FIG. 2.

FIG. 5 is a bottom view of the goggle of FIG. 2.

FIG. 6 is a left side view of the goggle of FIG. 2.

FIG. 7 is a right side view of the goggle of FIG. 2.

FIG. 8 is a rear perspective view of the goggle of FIG. 2.

FIG. 9 is a front perspective view of the goggle of FIG. 2.

FIG. 10 is a front perspective view of an illustrative lens of the goggle of FIG. 2.

FIG. 11 is a rear oblique view of the lens of FIG. 10.

FIG. 12 is a first front perspective view of the goggle of FIG. 2 with the lens, canister covers, and film rolls removed.

FIG. 13 is a second front perspective view of the goggle of FIG. 2 with the lens, canister covers, and film rolls removed.

FIG. 14 is a front perspective view of the goggle of FIG. 2, with the lens, second frame segment, canister covers, and film removed.

FIG. 15 is a front perspective view of the goggle of FIG. 2 with the canister covers removed.

FIG. 16 is a front perspective view of the goggle of FIG. 2 with the canister covers and the film rolls removed.

FIG. 17 is a front perspective view of the goggle of FIG. 16 showing an integral hook of the lens engaged with a pin of the goggle frame.

FIG. 18 is a front perspective view of the goggle of FIGS. 16-17 showing a latch mechanism of the goggle frame mated with a latch-receiving aperture of the lens.

FIG. 19 is a front perspective view of a latch mechanism of the goggle frame engaged with the lens of the goggle of FIG. 2.

FIG. 20 is a front view of the latch mechanism of the goggle of FIG. 2.

FIG. 21 is a front perspective view of a first integral film canister of the goggle of FIG. 2.

FIG. 22 is a front perspective view of the first integral film canister of FIG. 21 with a canister cover removed.

FIG. 23 is a front perspective view of a canister cover of the goggle of FIG. 2.

FIG. 24 is a rear perspective view of the canister cover of FIG. 23.

FIG. 25 is a front perspective view of the goggle of FIG. 2 with an illustrative mud visor coupled to the goggle frame in accordance with aspects of the present disclosure.

FIG. 26 is a front perspective view of the mud visor shown in FIG. 25.

FIG. 27 is a top view of the mud visor shown in FIG. 25.

FIG. 28 is a bottom view of the mud visor shown in FIG. 25.

FIG. 29 is a rear perspective view of the goggle of FIG. 2 with the illustrative mud visor shown in FIGS. 25-28 installed on the goggle frame.

FIG. 30 is rear perspective view of another illustrative example of the goggles of FIGS. 1 and 2 in accordance with aspects of the present teachings.

FIG. 31 is a rear perspective view of the goggle of FIG. 31 with a pair of rear latch locking members removed.

FIG. 32 is a front view of the goggle of FIGS. 30 and 31 with a lens removed from the goggle frame.

FIG. 33 is rear oblique view of an illustrative lens of the goggle of FIGS. 30-32.

FIG. 34 is a method of assembling a goggle in accordance with aspects of the present disclosure.

### DETAILED DESCRIPTION

Various aspects and examples of a goggle having integrated film canisters, as well as related methods, are described below and illustrated in the associated drawings. Unless otherwise specified, a goggle in accordance with the present teachings, and/or its various components, may contain at least one of the structures, components, functionalities, and/or variations described, illustrated, and/or incorporated herein. Furthermore, unless specifically excluded, the process steps, structures, components, functionalities, and/or variations described, illustrated, and/or incorporated herein in connection with the present teachings may be included in other similar devices and methods, including being interchangeable between disclosed embodiments. The following description of various examples is merely illustrative in nature and is in no way intended to limit the disclosure, its application, or uses. Additionally, the advantages provided by the examples and embodiments described below are illustrative in nature and not all examples and embodiments provide the same advantages or the same degree of advantages.

This Detailed Description includes the following sections, which follow immediately below: (1) Definitions; (2) Overview; (3) Examples, Components, and Alternatives; (4) Advantages, Features, and Benefits; and (5) Conclusion. The Examples, Components, and Alternatives section is further divided into subsections, each of which is labeled accordingly.

### Definitions

The following definitions apply herein, unless otherwise indicated.

“Comprising,” “including,” and “having” (and conjugations thereof) are used interchangeably to mean including but not necessarily limited to, and are open-ended terms not intended to exclude additional, unrecited elements or method steps.

Terms such as “first”, “second”, and “third” are used to distinguish or identify various members of a group, or the like, and are not intended to show serial or numerical limitation.

“AKA” means “also known as,” and may be used to indicate an alternative or corresponding term for a given element or elements.

“Elongate” or “elongated” refers to an object or aperture that has a length greater than its own width, although the width need not be uniform. For example, an elongate slot may be elliptical or stadium-shaped, and an elongate candlestick may have a height greater than its tapering diameter. As a negative example, a circular aperture would not be considered an elongate aperture.

“Coupled” means connected, either permanently or releasably, whether directly or indirectly through intervening components.

“Resilient” describes a material or structure configured to respond to normal operating loads (e.g., when compressed) by deforming elastically and returning to an original shape or position when unloaded.

“Rigid” describes a material or structure configured to be stiff, non-deformable, or substantially lacking in flexibility under normal operating conditions.

“Elastic” describes a material or structure configured to spontaneously resume its former shape after being stretched or expanded.

Directional terms such as “up,” “down,” “vertical,” “horizontal,” and the like should be understood in the context of the particular object in question. For example, an object may be oriented around defined X, Y, and Z axes. In those examples, the X-Y plane will define horizontal, with up being defined as the positive Z direction and down being defined as the negative Z direction.

In this disclosure, one or more publications, patents, and/or patent applications may be incorporated by reference. However, such material is only incorporated to the extent that no conflict exists between the incorporated material and the statements and drawings set forth herein. In the event of any such conflict, including any conflict in terminology, the present disclosure is controlling.

### Overview

In general, a goggle in accordance with the present teachings includes a goggle frame including a pair of integral canisters integrated with the goggle frame. The pair of integral canisters are configured to facilitate indexing a protective lens film across a front surface of a lens of the goggle. The lens of the goggle is configured to be releasably coupled to a front side of the goggle frame in a lens receiving area of the goggle frame. The goggle frame includes at least one latch mechanism that is configured to releasably engage a lateral end of the lens to secure the lens to the goggle frame.

### EXAMPLES, COMPONENTS, AND ALTERNATIVES

The following sections describe selected aspects of illustrative protective goggles as well as related systems and/or methods. The examples in these sections are intended for illustration and should not be interpreted as limiting the scope of the present disclosure. Each section may include one or more distinct embodiments or examples, and/or contextual or related information, function, and/or structure.

### A. Illustrative Protective Goggles

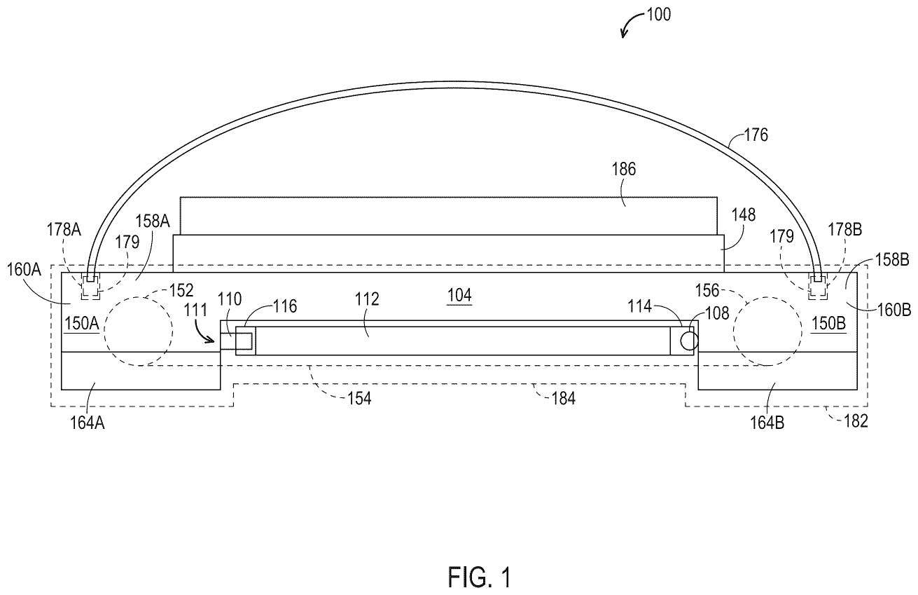

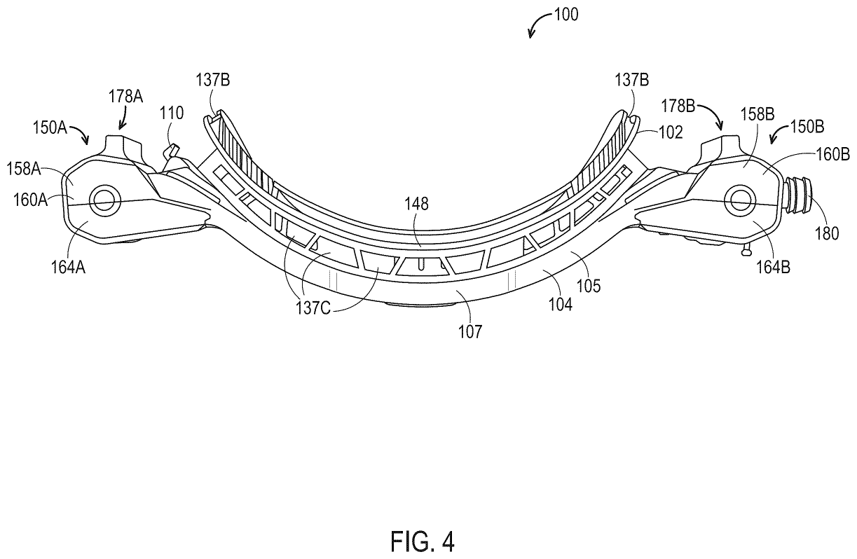

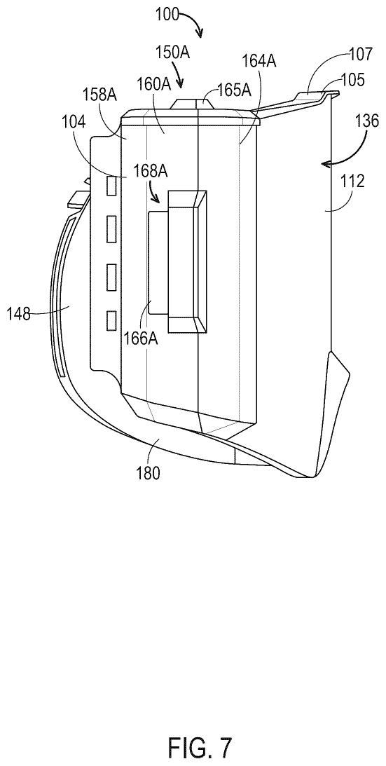

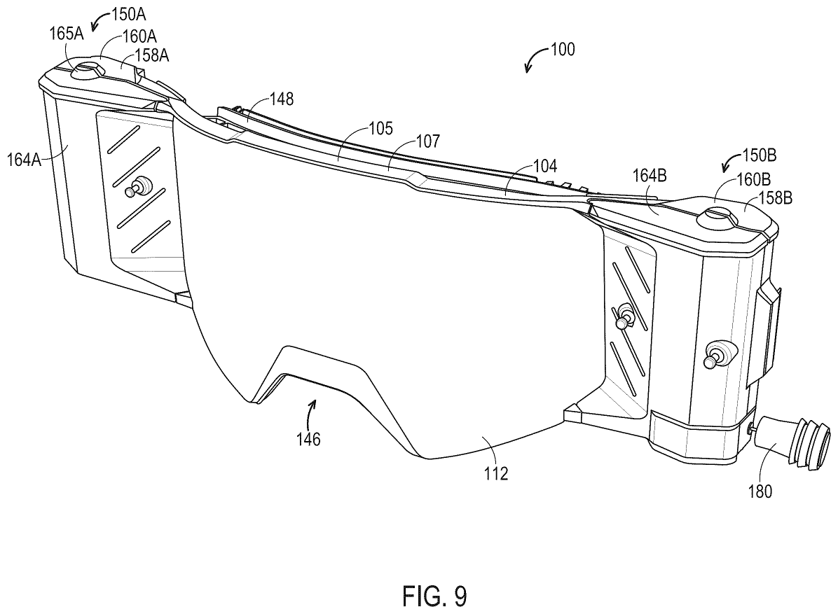

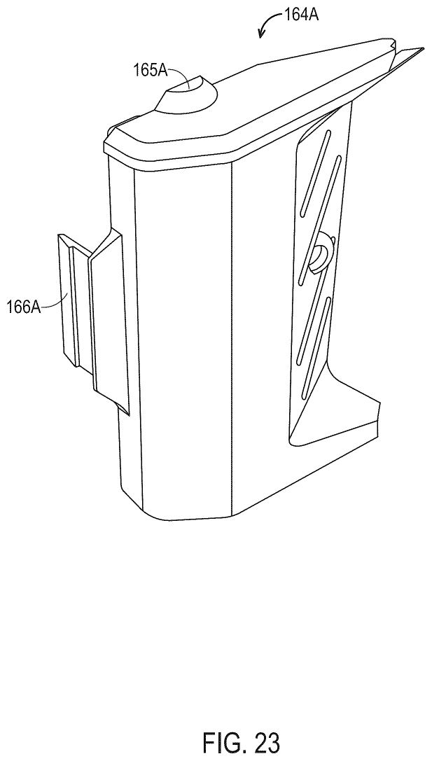

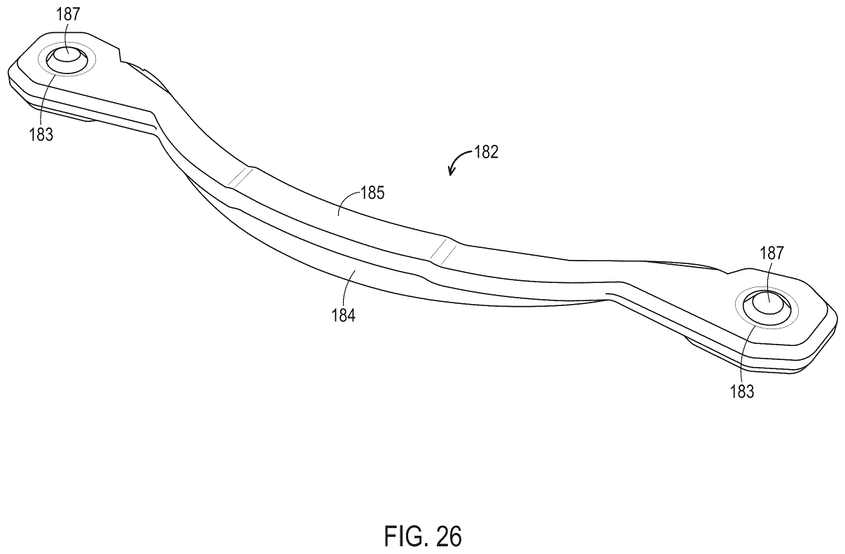

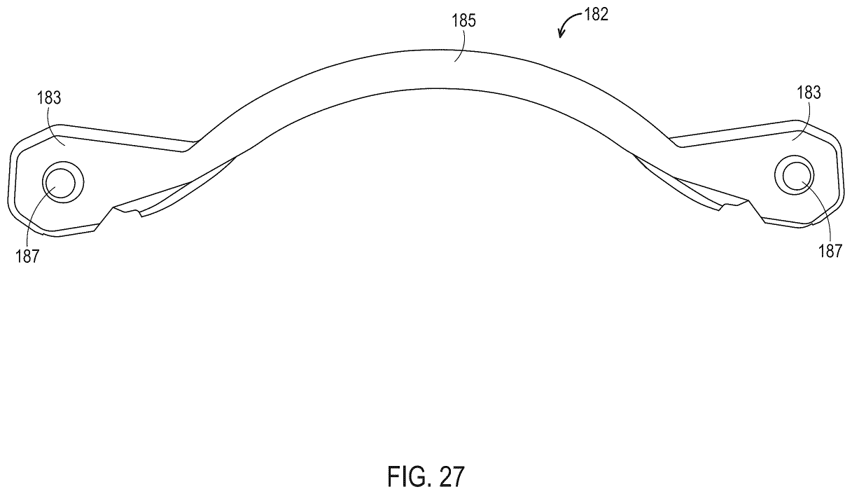

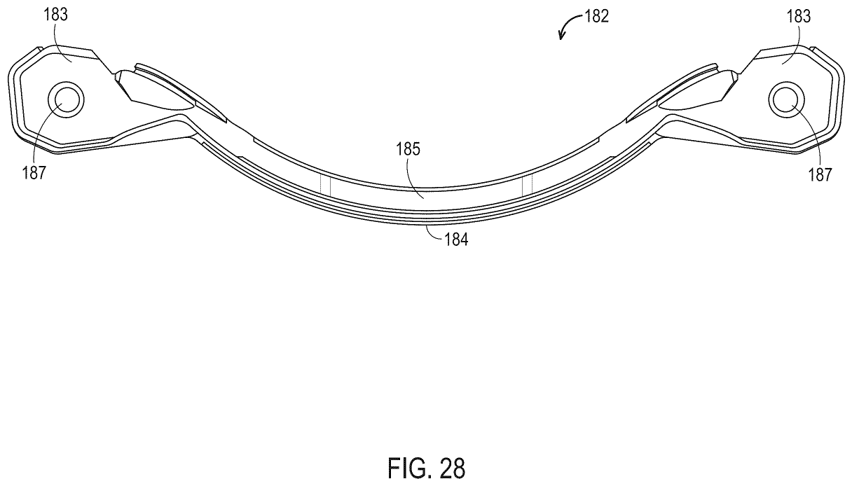

As shown in FIGS. 1-33, this section describes an illustrative protective goggle 100 (AKA protective sports goggle, goggle) for use in sports or activities, such as motocross, skiing, snowboarding, etc.

FIG. 1 illustrates a schematic top plan view of goggle 100. As shown in FIG. 1, goggle 100 includes a goggle frame 102 and a lens 112 configured to be releasably coupled to goggle frame 102. Goggle frame 102 is configured to provide a structural support for lens 112 and a roll-off film system of goggle 100.

Goggle frame 102 includes a lens receiving area 106 configured to receive lens 112 and a pair of integral film canisters 150A, 150B (AKA integral canisters, canisters) configured to receive respective rolls of lens film 154. For example, goggle frame 102 may include a first canister 150A (AKA a supply canister) configured to support and house a supply roll 152 of lens film 154 and a second canister 150B (AKA a take-up canister) configured to support and house a take-up roll 156 of lens film 154. Canisters 150A, 150B are disposed on opposing lateral sides (e.g., left and right sides) of lens receiving area 106 and are configured to facilitate indexing lens film 154 across a front surface of lens 112 during operation. Although lens film 154 is shown in FIG. 1 as spaced in front of lens 112, lens film 154 may contact the front surface of lens 112 when extending across the front surface of lens 112. Lens film 154 is configured to cover a substantial portion of the front surface of lens 112, such that lens film 154 protects the front surface of lens 112 from becoming covered in dirt and/or other debris, during use. When lens film 154 becomes filled with dirt and/or other debris, the used lens film 154 may be conveyed across lens 112 from supply roll 152 to take-up roll 156 and replaced by a new, clean section of lens film 154 from supply roll 152. As discussed further below, goggle 100 includes a pull-cord ratchet mechanism 180 and/or any other suitable mechanism that is configured to convey lens film 154 across the front surface of lens 112 from supply roll 152 to take-up roll 156.

In some examples, goggle frame 102 includes a first frame segment 104, a second frame segment 148, and/or a face contacting layer 186 (e.g., a foam layer or foam portion). As shown in FIG. 1, in some examples, first frame segment 104 comprises an integral, single-piece structure that forms and/or defines lens receiving area 106 and/or that forms at least a portion of each of integral canisters 150A, 150B (e.g., a rear portion). For example, respective rear portions 160A, 160B of each of integral canisters 150A, 150B may be formed by an integral part 158A, 158B of first frame segment 104 of goggle frame 102. Additionally, or alternatively, respective front portions and/or any other suitable portions of integral canisters 150A, 150B may be formed by integral parts 158A, 158B of first frame segment 104. Additionally, or alternatively, in some examples, integral canisters 150A, 150B may be integral with second frame segment 148 in addition to first frame segment 104 or rather than first frame segment 104. For example, first and/or second integral parts of second frame segment 148 may form any suitable portion of integral canisters 150A, 150B, e.g., a rear portion and/or a front portion of integral canisters 150A, 150B.

In some examples, as shown in FIG. 1, a head strap 176 of goggle 100 is configured to be coupled at each end to first frame segment 104. For example, head strap 176 may be coupled to a rear side of the integral parts 158A, 158B of first frame segment 104 that also form the respective rear portions 160A, 160B of integral canisters 150A, 150B. Explained in other words, goggle frame 102 may include a single-piece first frame segment 104 that forms lens receiving area 106 configured to receive lens 112, forms respective rear portions of each of integral film canisters 150A, 150B, and that is operatively coupled to head strap 176. In some examples, each of integral film canisters 150A, 150B includes a respective canister cover 164A, 164B configured to be removably coupled to first frame segment 104 to enclose supply roll 152 and take-up roll 156 of lens film 154 within integral canisters 150A, 150B.

In some examples, goggle frame 102 comprises second frame segment 148 coupled to a rear side of first frame segment 104 in any suitable manner, such that first frame segment 104 and second frame segment 148 are configured to remain coupled to each other permanently. Second frame segment 148 may connect first frame segment 104 to face contacting layer 186 of goggle frame 102. In some examples, face contacting layer 186 comprises foam and/or any other suitable resilient material(s) configured to provide a comfortable surface for contacting the user's face during use.

Lens 112 may be configured to be coupled to goggle frame 102 in any suitable manner, such that lens 112 is securely retained (e.g., held in a fixed position) relative to goggle frame 102 during operation and such that lens 112 is selectively removable from goggle frame 102. In some examples, lens 112 is configured to be operatively received and retained in and/or on lens receiving area 106 of goggle frame 102. In some examples, goggle frame 102 includes one or more structures and/or features disposed in or proximate to lens receiving area 106 that are configured to facilitate coupling and securing lens 112 to goggle frame 102. For example, goggle frame 102 may comprise a pin 108 disposed at a first lateral end of lens receiving area 106 and a latch mechanism 111 disposed at a second lateral end of lens receiving area 106, as discussed further below.

As shown in FIG. 1, in some examples, a first lateral end of lens 112 is configured to be pivotably coupled to goggle frame 102, e.g., pivotably coupled to pin 108 disposed at the first lateral end of lens receiving area 106. In such examples, lens 112 may be configured to be pivoted relative to goggle frame 102 (e.g., about pin 108), such that a second lateral end of lens 112 is positioned proximate goggle frame 102, e.g., such that the second lateral end of lens 112 is received in lens receiving area 106. In some examples, the second lateral end of lens 112 is configured to be latched to goggle frame 102 (e.g., using latch mechanism 111) to secure lens 112 to goggle frame 102 in lens receiving area 106.

In some examples, lens 112 includes one or more structures and/or features configured to facilitate coupling and securing lens 112 to goggle frame 102. For example, lens 112 may include a hook 114 (e.g., an integral hook) disposed at the first lateral end of lens 112 and/or a latch-receiving aperture 116 disposed at the second lateral end of lens 112. In some examples, hook 114 is configured to operatively engage pin 108 to pivotably couple lens 112 to goggle frame 102. In some examples, latch-receiving aperture 116 is configured to be operatively mated (e.g., receive) with a latch locking member 110 of latch mechanism 111 to secure lens 112 to goggle frame 102.

In some examples, latch locking member 110 of latch mechanism 111 is configured to be selectively engaged and disengaged with lens 112. For example, latch locking member 110 may be configured to be transitioned between a locked position and an unlocked position. When latch locking member 110 is in the locked position, latch locking member 110 is configured to operatively mate (e.g., insert into) latch-receiving aperture 116 of lens 112 to secure lens 112 to goggle frame 102. When latch locking member 110 is in the unlocked position, latch locking member 110 is configured to be removed from latch-receiving aperture 116 and disengaged from lens 112, such that the second lateral end of lens 112 is permitted to be selectively removed from or inserted into lens receiving area 106 of goggle frame 100.

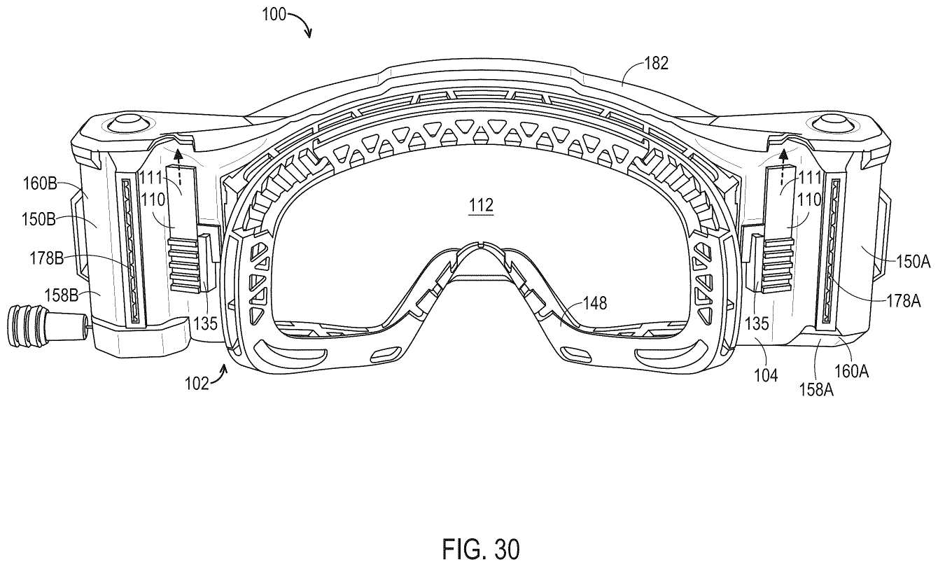

Alternatively, in some examples, goggle frame 102 may include a pair of latch mechanisms 111 configured to releasably engage the first and second lateral ends of lens 112 to secure lens 112 to goggle frame 102, e.g., as shown in the example of FIGS. 30-33. In such examples, lens 112 is configured to be latched at each end to goggle frame 102, rather than being pivotably coupled to goggle frame 102 at one end and latched to goggle frame 102 at the other end. In some such examples, lens 112 may include a pair of latch-engagement tabs 135 and/or any other suitable structures disposed at opposing lateral ends of lens 112 that are configured to be selectively engaged by a respective one of the pair of latch mechanisms 111 of goggle frame 102.

As shown in FIG. 1, in some examples, goggle 100 includes a mud visor 182 configured to be selectively coupled to and decoupled from an upper surface or upper side of goggle frame 102. In some examples, mud visor 182 is configured to extend over and/or cover the upper surface of first frame segment 104 of goggle frame 102 including extending over and covering integral canisters 150A, 150B of goggle frame 102. Mud visor 182 includes a downward extending mud flap 184 that is configured to extend in front of at least an upper portion of a front surface of lens 112 and an upper edge of lens film 154 that extends across the front surface of lens 112. Mud flap 184 is configured to prevent dirt and debris from entering between lens film 154 and the front surface of lens 112 during use. Mud visor 182 may comprise a deformable material configured to permit selective attachment and detachment of mud visor 182 to goggle frame 102, e.g., in a press-fit arrangement.

As shown in FIG. 1, goggle 100 includes a head strap 176 coupled to goggle frame 102 and configured to extend around the user's head to secure goggle frame 102 to the user. Head strap 176 may comprise any suitable length-adjustable strip of material configured to secure goggle 100 to the user's head. In some examples, head strap 176 is elastic. In some examples, head strap 176 is coupled at each end to first frame segment 104 of goggle frame 102 in a manner configured to prevent detachment of head strap 176 from first frame segment 104 during use.

In some examples, a suitable head strap attachment mechanism for coupling head strap 176 to first frame segment 104 of goggle 100 is described in U.S. Nonprovisional patent application Ser. No. 16/824,525. For example, first and second vertical recesses 178A, 178B may be formed in the rear side of first and second integral parts 158A, 158B of first frame segment 104 that form rear portions 160A, 160B of first and second integral canisters 150A, 150B. Each end of head strap 176 may be disposed within a respective cage 179 that is configured to be received within one of the first and second vertical recesses 178A, 178B. Each cage 179 may comprise a metal structure (e.g., folded sheet metal) that is fixed to a respective end of head strap 176, e.g., using one or more fasteners. Each cage 179 is configured to be received and secured within a respective one of first and second vertical recesses 178A, 178B, such that each end of head strap 176 is secured to first frame segment 104. In some examples, first frame segment 104 and/or cages 179 comprise one or more retainment features configured to engage with each other to prevent cages 179 and head strap 176 from being removed from first and second vertical recesses 178A, 178B. For example, cages 179 may comprise one or more detents and first frame segment 104 may comprise one or more corresponding openings and/or depressions within first and second vertical recesses 178A, 178B configured to engage with the detents to prevent cages 179 from being removed from first and second vertical recesses 178A, 178B.

In general, as discussed herein, the rearward side of the components of goggle 100 refers to the side that is configured to be disposed closer to the user's face when goggle is worn by the user. The forward or front side of the components of goggle 100 refers to the side that is configured to be disposed further from the user's face when the goggle is worn. For example, second frame segment 148 is disposed rearward of first frame segment 104 and is coupled to a rear side of first frame segment 104, such that second frame segment 148 is configured to be positioned closer to the user's face when goggle 100 is worn. Lens 112 is coupled to a front side or forward-facing side of first frame segment 104. Likewise, components that extend or project rearward from a structure extend from that structure generally toward the user's face when goggle 100 is worn and components that extend or project forward from a structure extend from that structure generally away from the user's face when goggle 100 is worn. As used herein, the lateral sides of goggle 100 refer to the left and/or right sides of goggle 100. For example, first and second integral canisters 150A, 150B are disposed at opposing lateral ends (e.g., right and left lateral ends) of goggle 100 and first frame segment 104.

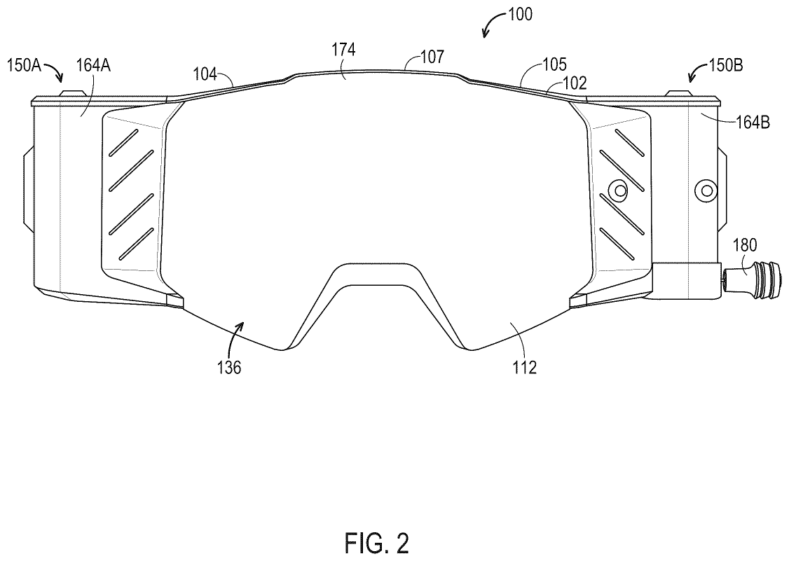

FIGS. 2-9 illustrate various views of an example of goggle 100 schematically illustrated in FIG. 1. As shown in FIGS. 2-9, goggle 100 includes a goggle frame 102 with a pair of integrated canisters 150A, 150B and a lens 112 operatively coupled to a front side of goggle frame 102.

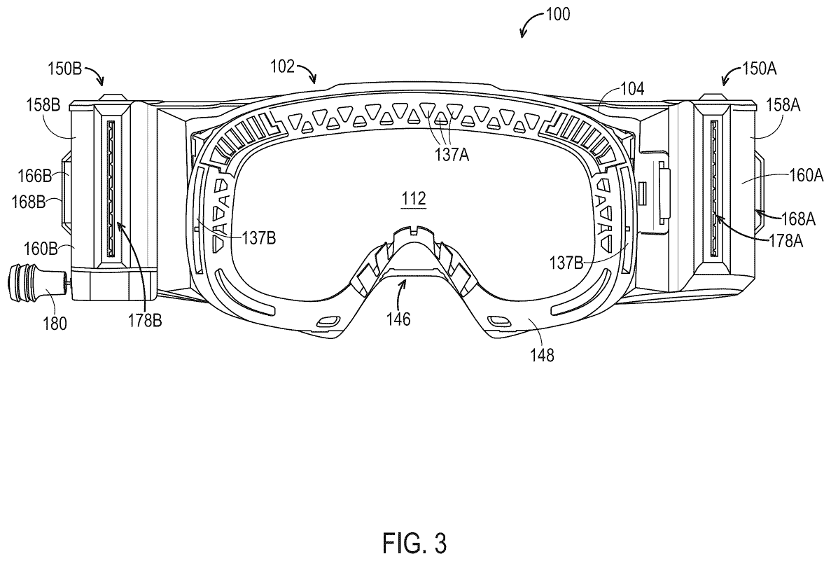

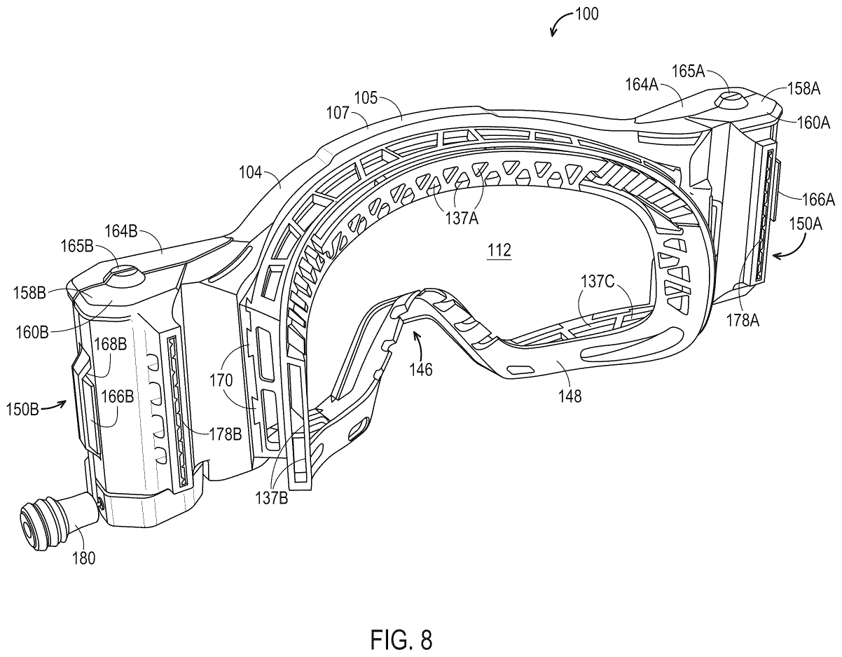

As shown most clearly in FIGS. 3-5 and 8, goggle frame 102 includes a first frame segment 104 and a second frame segment 148 operatively coupled to a rear side of first frame segment 104, i.e., the side closer to the user's face when the goggle is worn. Second frame segment 148 may be coupled to first frame segment 104 in any suitable manner configured to keep the first and second frame segments attached permanently. For example, as shown most clearly in FIG. 8, second frame segment 148 is coupled to first frame segment 104 by a plurality of dovetail joints 170.

First frame segment 104 and second frame segment 148 may comprise any suitable materials configured to be secured to the user's face (e.g., using head strap 176) and configured to support lens 112. In some examples, first frame segment 104 and second frame segment 148 comprise different materials. For example, first frame segment 104 may be more rigid than second frame segment 148 and/or second frame segment may be more compliant or resilient than first frame segment.

In some examples, goggle 100 includes a face contacting portion comprising a resilient material or resilient layer that is configured to provide a comfortable surface that contacts the user's face when goggle 100 is worn by the user. In some examples, the face contacting portion comprises a foam layer. The foam layer and/or any other suitable face contacting portion of goggle 100 is coupled to a rear side of second frame segment 148, such that the face contacting portion or layer is configured to contact the user's face when the goggle 100 is worn.

As shown in FIGS. 3-5 and 8, in some examples, second frame segment 148 includes a lattice structure that defines or includes a plurality of openings 137A-C and/or through-holes. Second frame segment 148 may have any suitable number and arrangement of openings 137A-C. In some examples, plurality of openings 137A-C are configured to improve airflow and ventilation of goggle 100 and/or permit moisture to be released from goggle 100. For example, the plurality of openings 137A-C may include one or more vent openings 137C that are configured to improve ventilation and/or airflow through goggle 100 when worn by the user. In some examples, as shown in FIGS. 3 and 8, the plurality of openings may include one or more rear openings 137A formed on the rear surface of second frame segment 148. Rear openings 137A formed in the rear surface of second frame segment 148 may be configured to receive sweat and/or other moisture from the foam layer or other face contacting portion that is coupled to the rear surface of second frame segment 148. For example, the foam layer or other face contacting portion may be coupled to the rear surface of second frame segment 148, such that rear openings 137A interface with the face contacting layer and are configured to receive moisture from the face contacting layer. In some examples, the sweat and/or other moisture received through rear openings 137A formed in the rear surface of second frame segment 148 is permitted to exit second frame segment 148 and goggle 100 through other ones of the plurality of openings and/or through-holes formed in second frame segment 148. For example, second frame segment 148 may include one or more gutter openings 137B oriented generally transverse to rear openings 137A that are configured to permit moisture received through rear openings to exit second frame segment 148, e.g., via gravity. Gutter openings 137B may be disposed on top, bottom, and/or lateral sides of second frame segment 148. Collectively the plurality of openings 137A-C of second frame segment 148 are configured to improve airflow and ventilation of goggle 100, receive sweat and/or other moisture from a face contacting portion (e.g., foam layer) of goggle 100, and/or permit the sweat and/or other moisture to exit second frame segment 148 and goggle 100 during use.

In some examples, goggle 100 includes first and second integral film canisters 150A, 150B disposed at opposing lateral ends of goggle frame 102 and integrated with goggle frame 102. For example, at least a portion of first and second integral film canisters 150A, 150B may be formed by respective integral parts of first frame segment 104, second frame segment 148, and/or any other suitable structure(s) of goggle frame 102. For example, rear portions and/or front portions of first and second integral film canisters 150A, 150B may be formed by integral parts and/or integral portions of first frame segment 104 and/or second frame segment 148.

In some examples, each integral film canister 150A, 150B includes a respective canister cover 164A, 164B configured to be operatively coupled to the integral part of goggle frame 102 that forms at least the portion of integral film canister 150A, 150B. For example, respective rear portions of integral film canisters 150A, 150B may be formed by integral parts of first frame segment 104 and/or second frame segment 148, and in such examples, canister covers 164A, 164B may be configured to form respective front portions of integral film canisters 150A, 150B. Alternatively, in some examples, respective front portions of integral film canisters 150A, 150B may be formed by integral parts of first frame segment 104 and/or second frame segment 148, and in such examples, canister covers 164A, 164B may be configured to form respective rear portions of integral film canisters 150A, 150B. Explained in other words, in some examples, canister covers 164A, 164B are configured to be releasably coupled to a front side or a rear side of the integral parts of goggle frame 102 that form the respective portions of integral film canisters 150A, 150B, such that canister covers 164A, 164B form removable front and/or rear portions of integral film canisters 150A, 150B.

The components and/or structures of goggle frame 102 including the integral parts of goggle frame 102 that form at least the respective portions of integral film canisters 150A, 150B may comprise any suitable material(s) configured to support lens 112 and configured to be coupled to a user's face. For example, structure(s) of goggle frame 102 may comprise a molded plastic, composite, and/or any other suitable material(s). First frame segment 104 and second frame segment 148 may comprise a same material and/or may comprise different materials than each other. In some examples, first frame segment 104 comprises a material having greater rigidity and/or stiffness than second frame segment 148.

In some examples, as shown in FIGS. 2-9, first frame segment 104 of goggle 100 includes first and second integral parts 158A, 158B that are configured to form respective portions of first and second integral canisters 150A, 150B of goggle frame 102, such that canisters 150A, 150B are integrated into goggle frame 102. For example, first and second integral parts 158A, 158B of first frame segment 104 may form rear portions of first and second integral canisters 150A, 150B, respectively.

First and second canisters 150A, 150B are each configured to support a respective roll of lens film 154. As discussed further below, first canister 150A (AKA supply canister 150A) is configured to support and house a supply roll 152 of lens film 154 and second canister 150B (AKA take-up canister 150B) is configured to support and house a take-up roll 156 of lens film 154. Lens film 154 is configured to be selectively conveyed across the front surface of lens 112 from supply roll 152 to take-up roll 156, e.g., by actuating pull-cord ratchet mechanism 180. In the example of goggle 100 shown in FIGS. 2-24, first canister 150A that supports supply roll 152 is disposed at a right lateral end of first frame segment 104 and second canister 150B that houses take-up roll 156 is disposed at a left lateral end of first frame segment 104. In this example, lens film 154 is configured to be conveyed right to left across the front surface of lens 112. Alternatively, first canister 150A that houses supply roll 152 may be disposed at the left lateral end of first frame segment 104 and second canister 150B that houses take-up roll 156 may be disposed at the right lateral end of first frame segment 104. In such alternative examples, lens film 154 is configured to be conveyed from left to right across the front surface of lens 112.

As shown in FIG. 3, in some examples, a head strap 176 is configured to be operatively coupled to first and second integral parts 158A, 158B of first frame segment 104. For example, a first end of the head strap 176 may be received in and/or plugged into first integral part 158A of first frame segment 104 that forms a rear portion of first integral canister 150A and a second end of head strap 176 may be received in and/or plugged into a second integral part 158B of first frame segment 104 that forms a rear portion of a second integral canister 150B of goggle frame 102. In some examples, as shown in FIG. 3, a first vertical recess 178A is formed in first integral part 158A and a second vertical recess 178B is formed in second integral part 158B of first frame segment 104 and each vertical recess 178A, 178B is configured to receive and retain a respective end of head strap 176.

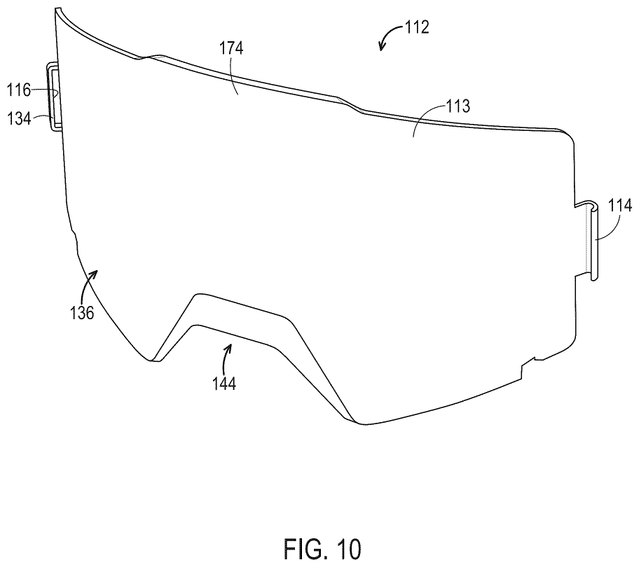

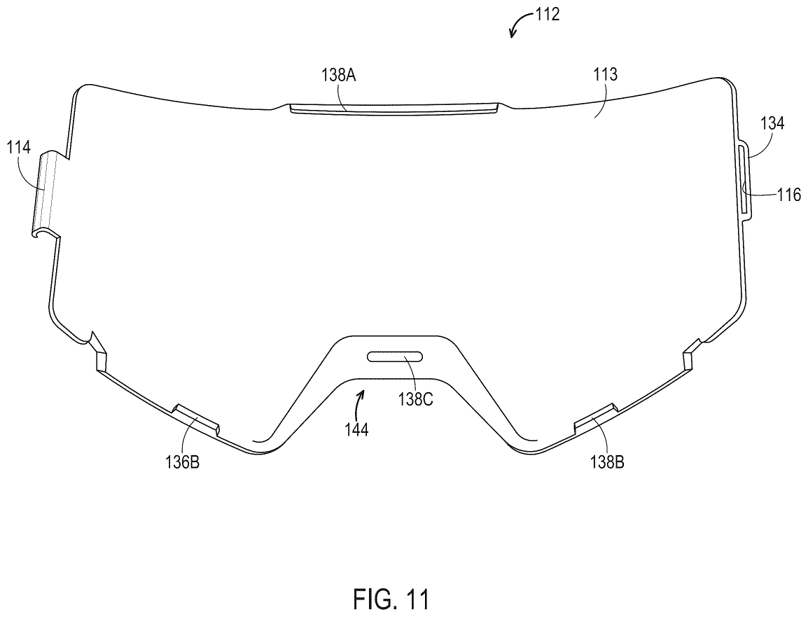

FIGS. 10-11 illustrate a front perspective view and a rear perspective view of lens 112, respectively. As shown in FIGS. 10-11, lens 112 comprises a monolithic structure configured to cover both eyes of the user when coupled to goggle frame 102. Lens 112 may have any suitable size and/or shape, such as a cylindrical contour or curvature, and may be constructed using any suitable method, such as an injection-molded plastic. In some examples, lens 112 is substantially rigid and/or configured to hold its overall shape during use and when coupled to goggle frame 102.

Lens 112 includes a plurality of engagement features that are configured to operatively engage goggle frame 102 (e.g., first frame segment 104) to facilitate coupling and securing lens 112 to goggle frame 102. In some examples, lens 112 includes a first engagement feature disposed on a first lateral side of lens 112 that is configured to facilitate pivotably coupling lens 112 to first frame segment 104 and a second engagement feature disposed on a second lateral side of lens 112 that is configured to facilitate latching the second lateral side of lens 112 to first frame segment 104.

For example, as shown in FIG. 10, lens 112 includes an integral hook 114 disposed on the first lateral side of lens 112 and a latch-receiving aperture 116 disposed on the second lateral side of lens 112. Integral hook 114 includes a proximal portion that extends laterally outward from the first lateral side of a main-body portion 113 of lens 112 and a curved or hooked distal portion that curves forward from the proximal portion of integral hook 114. Integral hook 114 is configured to facilitate pivotably coupling lens 112 to first frame segment 104 of goggle frame 102, as discussed further below.

Latch-receiving aperture 116 of lens 112 is formed in an integral tab 134 projecting forward from a front surface 136 of main-body portion 113 of lens 112, such that latch-receiving aperture 116 is disposed in front of a front surface of main-body portion 113 of lens 112. Explained in other words, latch-receiving aperture 116 is defined between integral tab 134 and the front surface of main-body portion 113 of lens 112. Both integral tab 134 and integral hook 114 comprise integral portions of lens 112. Explained in other words, lens 112 comprises a monolithic structure comprising main-body portion 113, integral tab 134, and integral hook 114, which are formed together as a single piece, e.g., a single injection-molded structure.

As shown in FIG. 11, in some examples, lens 112 includes one or more engagement protrusions 138A-C (AKA rearward projections) projecting rearward from lens 112 that are configured to operatively engage goggle frame 102. Lens 112 may include any suitable number and arrangement of engagement protrusion(s) 138A-C. In some examples, engagement protrusions 138A-C, like integral tab 134 and integral hook 114, are integral with lens 112. Explained in other words, lens 112 may comprise a single-piece, monolithic structure comprising main-body portion 113, integral tab 134, integral hook 114, and engagement protrusion(s) 138A-C.

In some examples, lens 112 includes one or more engagement protrusions 138A-C disposed proximate an upper or top edge of lens 112 (AKA top engagement protrusions), one or more engagement protrusions 138A-C disposed proximate a lower or bottom edge of lens 112 (e.g., bottom engagement protrusions), and/or one or more engagement protrusions 138A-C disposed proximate a lower nose cutout 172 of lens 112 (AKA nose engagement protrusions). For example, as shown in FIG. 11, engagement protrusion(s) 138A-C of lens 112 may include a top engagement protrusion 138A extending rearward from the upper edge of lens 112, a pair of bottom engagement protrusions 138B extending rearward from the bottom edge of lens 112, and a nose engagement protrusion 138C extending rearward from the bottom edge of lens 112 proximate a lower nose cutout 172 of lens. As discussed below, engagement protrusion(s) 138A-C are configured to fit into or under corresponding engagement pockets and/or gaps of goggle frame 102 to prevent lens 112 from moving relative to goggle frame 102, to prevent unwanted flexing of goggle frame 102 during use, and/or to operatively align lens 112 on goggle frame 102.

In some examples, lens 112 includes a lower nose cutout 172 formed in a bottom edge of lens 112 and/or a raised central portion 174 formed in an upper edge of lens 112. In some examples, raised central portion 174 extends upward from lateral portions of the upper or top edge of the lens 112. In some examples, as shown in FIG. 11, a respective one of engagement protrusions 138A extends rearward from raised central portion 174 and a respective one of engagement protrusions 138C extends rearward directly above lower nose cutout 172.

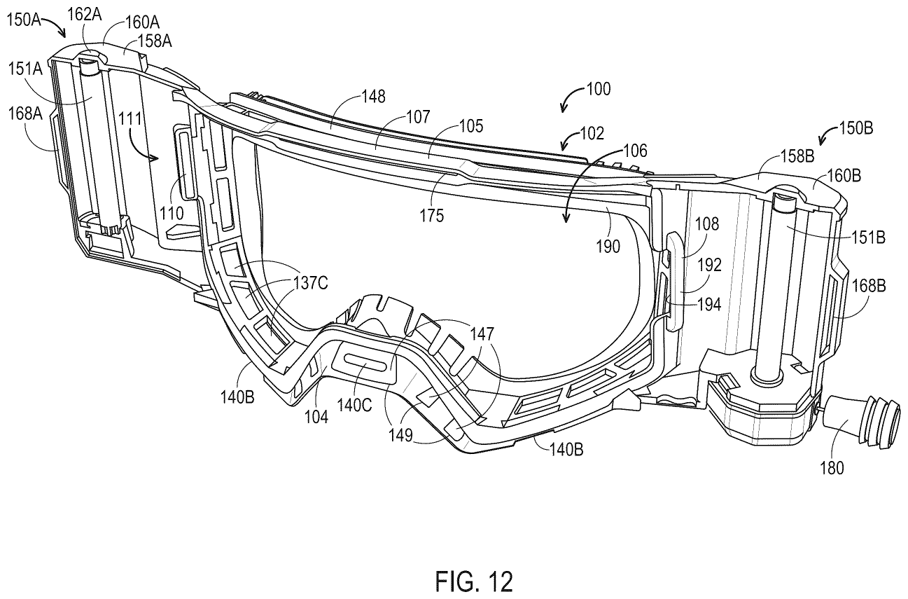

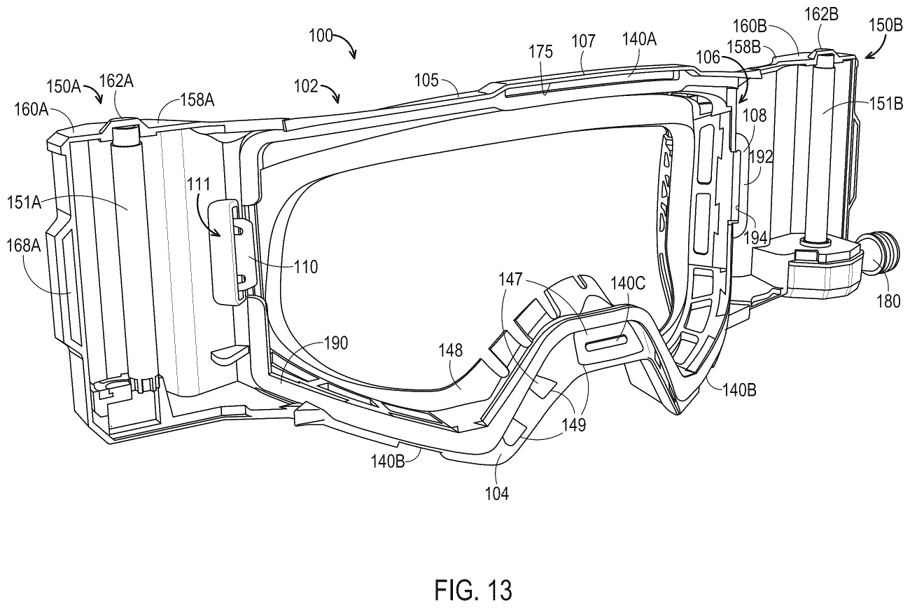

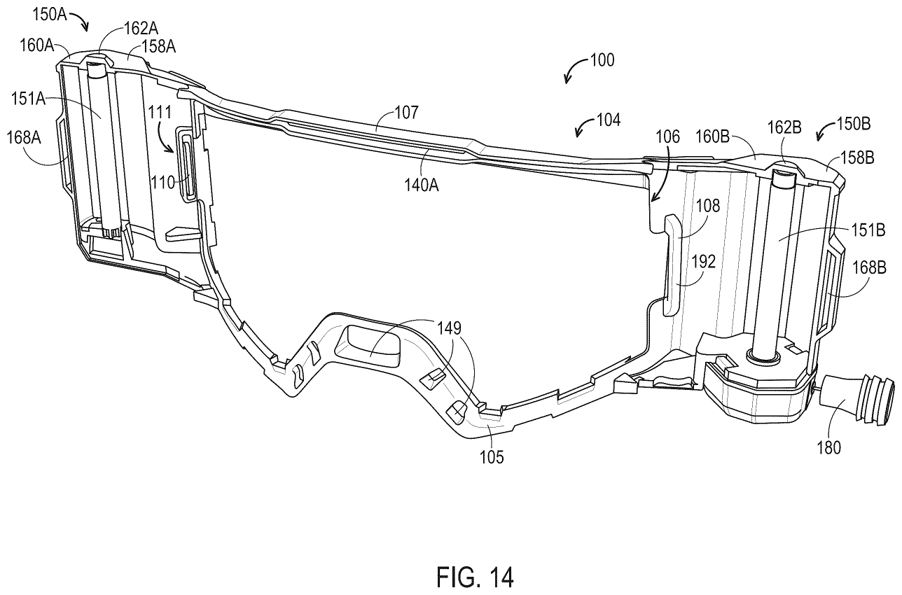

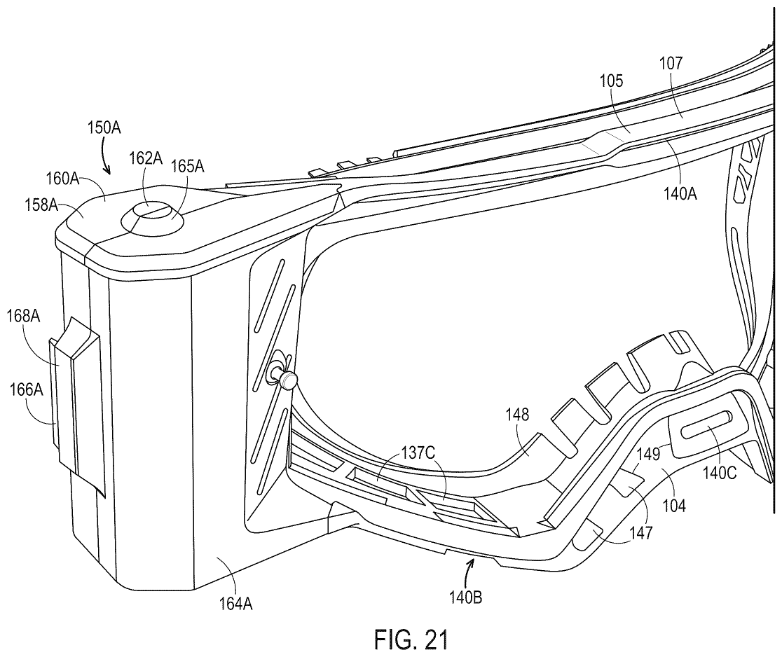

FIGS. 12-14 illustrate various views of goggle frame 102 with lens 112 removed from goggle frame 102. FIGS. 12 and 13 illustrate front-side perspective views of goggle frame 102 including first frame segment 104 and second frame segment 148 coupled to the rear side of first frame. FIG. 14 illustrates a front-side perspective view of first frame segment 104 with second frame segment 148 removed. In FIGS. 12-14, first and second canister covers 164A, 164B are removed from first and second canisters 150A, 150B. Canister covers 164A, 164B are described with reference to FIGS. 21-24 further below.

As shown in FIGS. 12-14, first frame segment 104 forms a peripheral rim 105 that defines and/or forms a lens receiving area 106. Peripheral rim 105 is unbroken and extends around a complete periphery to form a closed loop forming lens receiving area 106. Peripheral rim 105 defines a central viewing opening through which a user may view an external environment when wearing goggle 100. Lens receiving area 106 is sized and/or shaped to selectively receive lens 112. In some examples, lens 112 is configured to contact, abut, and/or rest against a front surface of first frame segment 104 when lens 112 is received in lens receiving area 106. Explained in other words, rather than being received within a lens groove or lens cutout of goggle frame 102, lens 112 may be configured to rest on and/or against the front surface of first frame segment 104 that forms and/or defines lens receiving area 106. In some examples, as shown in FIGS. 12-14, an upper rim 107 or edge of peripheral rim 105 is configured to overhang an upper edge of lens 112 when lens 112 is received in lens receiving area 106. In some examples, a gasket 190 is coupled to a front side of first frame segment 104. Gasket 190 may extend partially or entirely around the periphery of lens receiving area 106. Gasket 190 may comprise any suitable structures and/or material(s) configured to contact lens 112 and/or to form a releasable seal with lens 112 when lens 112 is received in lens receiving area 106 of first frame segment 104.

In the example of FIGS. 12-14, first and second integral parts 158A, 158B of first frame segment 104 disposed on opposing lateral sides of lens receiving area 106 each form a respective portion (e.g., a rear portion) of first and second canisters 150A, 150B of goggle frame 102, such that first and second canisters 150A, 150B are integral with first frame segment 104. In some examples, first frame segment 104 and first and second integral parts 158A, 158B of first frame segment 104 that form the respective portions of first and second canisters 150A, 150B comprise a continuous monolithic structure inseparable other than by using destructive methods. For example, first frame segment 104 and integral parts 158A, 158B of first frame segment 104 that form the respective portions of first and second canisters 150A, 150B may be molded as a single piece and/or may comprise or consist of a same material. Alternatively, in some examples, first frame segment 104 and first and second integral parts 158A, 158B of first frame segment 104 that form the respective portions of first and second canisters 150A, 150B may be connected permanently in any suitable manner configured to keep first and second integral parts 158A, 158B permanently fastened to first frame segment 104, e.g., using one or more single-use mechanical fasteners, locking joints, adhesives, etc.

In some alternative examples, rather than comprising an integral part of first frame segment 104, canisters 150A, 150B may be configured to be selectively and repeatedly coupled to and removed from first frame segment 104 without causing damage to first frame segment 104 or canisters 150A, 150B. For example, canisters 150A, 150B may be configured to be removably coupled to the opposing lateral sides of first frame segment 104 using one or more reusable fasteners, mating structures, and/or using any other suitable methods.

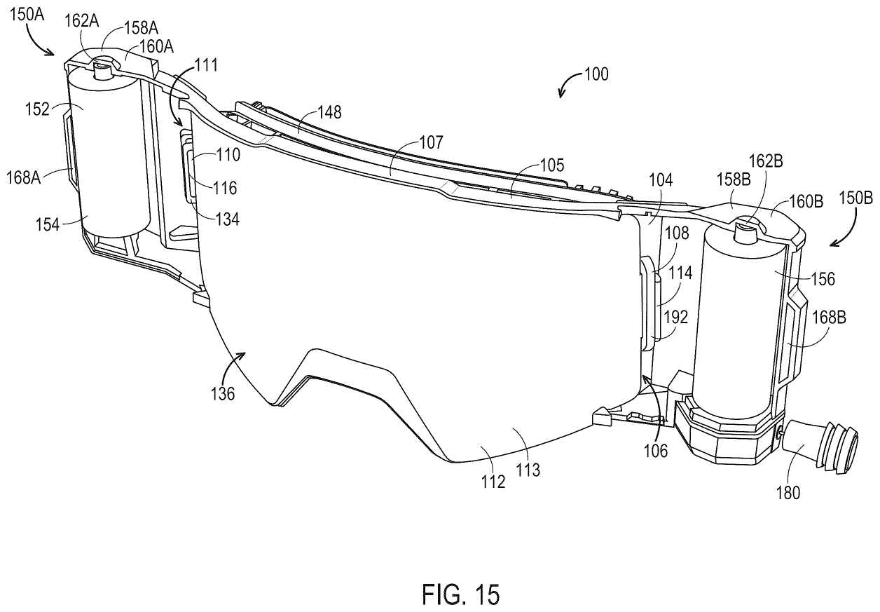

First and second integral parts 158A, 158B of first frame segment 104 that form at least the portion of first and second canisters 150A, 150B each include a respective spindle holder 162A, 162B configured to support respective spindles 151A, 151B on which a supply roll 152 and/or a take-up roll 156 of lens film 154 may be mounted, e.g., as shown in FIG. 15. Spindles 151A, 151B and spindle holders 162A, 162B are configured to permit selectively rotating supply roll 152 and take-up roll 156 within canisters 150A, 150B. During use, lens film 154 is configured to be indexed across a front surface of lens 112 from supply roll 152 to take-up roll 156. Goggle 100 may include any suitable mechanism configured to facilitate selectively indexing lens film 154 across the front surface of lens 112 from supply roll 152 to take-up roll 156. For example, as shown in FIGS. 12-14, goggle 100 includes a pull-cord ratchet mechanism 180 operatively coupled to a take-up spindle 151B on which take-up roll 156 is mounted, such that actuating (e.g., pulling) a pull cord of pull-cord ratchet mechanism 180 rotates take-up spindle 151B and take-up roll 156 mounted on take-up spindle 151B, which causes lens film 154 to be pulled across the front surface of lens 112 from supply roll 152 to take-up roll 156.

First frame segment 104 includes one or more engagement structures and/or features that are configured to operatively engage one or more corresponding engagement structures and/or features of lens 112 to facilitate removably coupling and securing lens 112 to first frame segment 104, e.g., within lens receiving area 106. For example, first frame segment 104 includes a pin 108 disposed at a first lateral end or side (e.g., a left side) of lens receiving area 106 and a latch mechanism 111 including a latch locking member 110 disposed at a second opposite lateral end or side (e.g., a right side) of lens receiving area 106. Pin 108 is configured to be operatively engaged with integral hook 114 of lens 112 to pivotably couple lens 112 to first frame segment 104, such that lens 112 is pivotable about pin 108. Latch locking member 110 of latch mechanism 111 is configured to mate with latch-receiving aperture 116 of lens 112 to secure and retain lens 112 in lens receiving area 106 of first frame segment 104.

In some examples, first frame segment 104 and/or second frame segment 148 include one or more engagement pockets 140A-C and/or engagement gaps that are configured to operatively receive engagement protrusions 138A-C of lens 112. For example, as shown in FIGS. 12 and 13, first frame segment 104 and/or second frame segment 148 include engagement pocket(s) 140A (e.g., top engagement pockets) disposed proximate an upper side or upper edge of lens receiving area 106 configured to receive an engagement protrusion 138A projecting rearward from the upper edge of lens 112, a pair of engagement pockets 140B (e.g., bottom engagement pockets) disposed proximate a bottom edge of lens receiving area 106 configured to receive a pair of engagement protrusions 138B projecting rearward from the bottom edge of lens 112, and an engagement pocket 140C (e.g., a nose engagement pocket) disposed proximate a nose bridge 146 of goggle frame 102 configured to receive an engagement protrusion 138C projecting rearward from lens 112 proximate nose cutout portion 144 of lens 112.

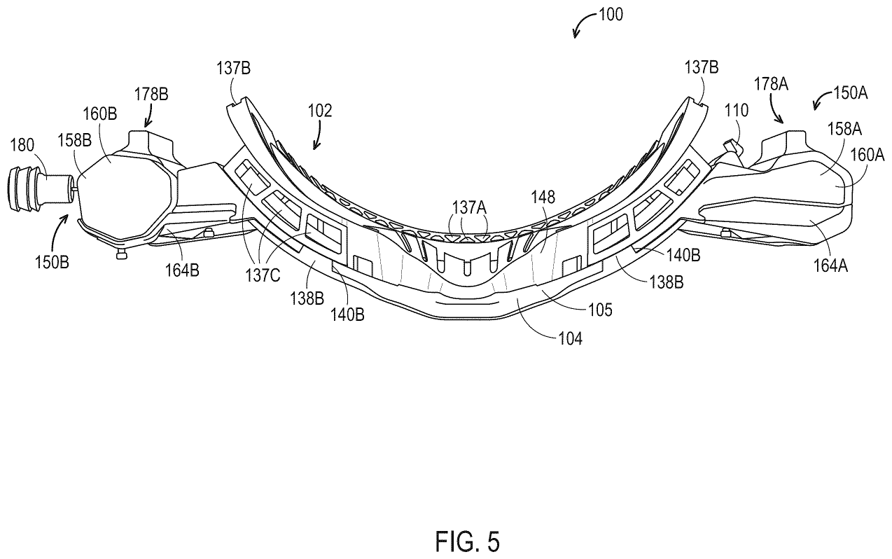

In some examples, as shown most clearly in FIG. 5, the pair of engagement pockets 140B disposed proximate the bottom edge of lens receiving area 106 each have an open lower end, such that respective bottom faces of the engagement protrusions 138B extending rearward from the bottom edge of lens 112 are exposed to an external environment when received in bottom engagement pockets 140B. Explained in other words, first frame segment 104 may comprise a pair of gaps formed in a lower edge of peripheral rim 105 that are configured to receive bottom engagement protrusions 138B extending rearward from the bottom edge of lens 112. In some examples, the top engagement pocket 140A disposed proximate an upper edge of lens receiving area 106 and the nose engagement pocket 140C disposed proximate nose bridge 146 of goggle frame 102 are each configured to fully enclose corresponding engagement protrusions 138A, 138C of lens.

In some examples, one or more of engagement pockets 140A-C are formed in first frame segment 104 and/or one or more of engagement pockets 140A-C are formed in second frame segment 148. For example, as shown in FIGS. 12-14, the top and bottom engagement pockets 140A, 140B formed proximate the upper edge and bottom edge of lens receiving area 106 are formed in first frame segment 104 and the nose engagement pocket 140C formed in nose bridge 146 of goggle frame 102 is formed in second frame segment 148. In some examples, as shown in FIG. 14, first frame segment 104 includes one or more first-frame openings 149 that are configured to receive respective portions 147 of second frame segment 148 to facilitate coupling second frame segment 148 to first frame segment 104. One of first-frame openings 149 is formed in a nose-bridge portion of first frame segment 104. As shown in FIGS. 12-13, a respective portion 147 of second frame segment 148 that extends through first-frame opening 149 at nose-bridge portion of first frame segment 104 defines the engagement pocket 140C formed in nose bridge 146 of goggle frame 102. As such, the corresponding nose engagement protrusion 138C projecting rearward from lens 112 proximate nose-cutout portion 144 of lens 112 is configured to be received in the engagement pocket 140C that is formed in second frame segment 148.

Engagement between engagement protrusions 138A-C extending rearward from lens 112 and engagement pockets 140A-C formed in first frame segment 104 and/or second frame segment 148 is configured to align lens 112 in lens receiving area 106, prevent undesired movement of lens 112 relative to goggle frame 102 when lens 112 is operatively coupled to goggle frame 102, and/or prevent undesired flexing of goggle frame 102 (e.g., first frame segment 104) during operation. For example, engagement between engagement protrusions 138A-C and engagement pockets 140a-C may be configured to prevent lens 112 from becoming dislodged from goggle frame 102 or improperly aligned with goggle frame 102 during use.

FIGS. 15-18 illustrate lens 112 operatively coupled to goggle frame 102. Lens 112 is configured to be coupled to goggle frame 102 by engaging integral hook 114 disposed at the first lateral end of lens 112 with pin 108 of first frame segment 104, pivoting lens 112 about pin 108, such that the second lateral end of lens 112 is positioned proximate first frame segment 104 and is disposed in lens receiving area 106 of first frame segment 104, and latching the second lateral end of lens 112 to goggle frame 102 using latch mechanism 111 of goggle frame 102. When lens 112 is pivoted about pin 108 to position the second lateral end of lens 112 in lens receiving area 106, engagement protrusions 138A-C of lens 112 are operatively inserted into corresponding engagement pockets 140A-C of goggle frame 102 to ensure proper alignment and prevent undesired flexing of goggle frame 102 during use.

When integral hook 114 is engaged with pin 108 and latch mechanism 111 is engaged with the second lateral end of lens 112 as shown in FIGS. 15-18, lens 112 is securely coupled to goggle frame 102, such that lens 112 is held in a fixed position and prevented from becoming dislodged from goggle frame 102 during use. Lens 112 is selectively removable from goggle frame 102 by disengaging latch mechanism 111 from the second lateral end of lens 112, pivoting lens 112 about pin 108 to remove the second lateral end of lens 112 from lens receiving area 106, and disengaging integral hook 114 of lens 112 from pin 108.

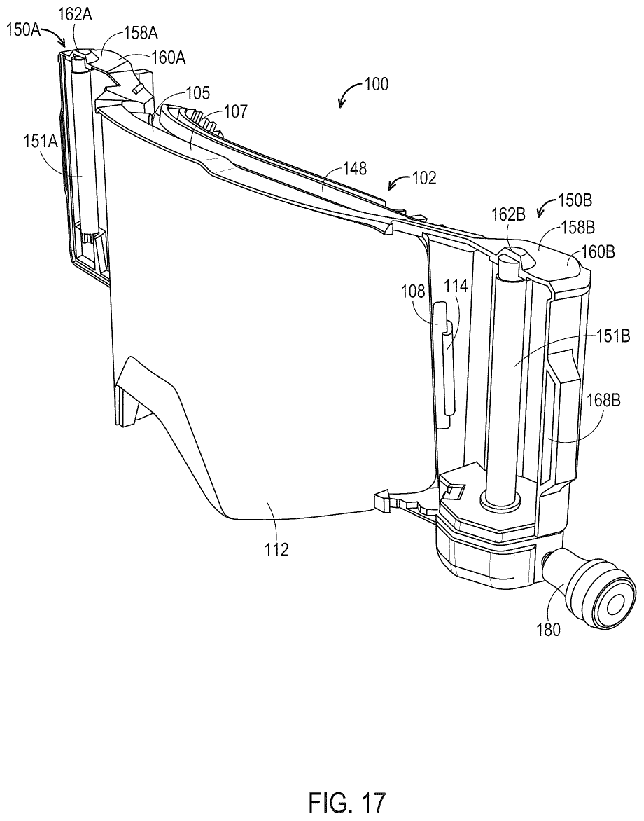

Pin 108 may comprise any suitable structures configured to form a pivoting joint with integral hook 114 of lens 112. For example, as shown most clearly in FIGS. 13 and 14, pin 108 may comprise an integral frame tab 192 projecting forward from first frame segment 104 at the first lateral end of lens receiving area 106 to define a hook-receiving aperture 194. As shown in FIG. 17, integral hook 114 of lens 112 is configured to be operatively received within hook-receiving aperture 194 of pin 108 to form a pivotable joint permitting lens 112 to be pivoted about pin 108. In some examples, pin 108 (e.g., integral frame tab 192) is an integral part of first frame segment 104.

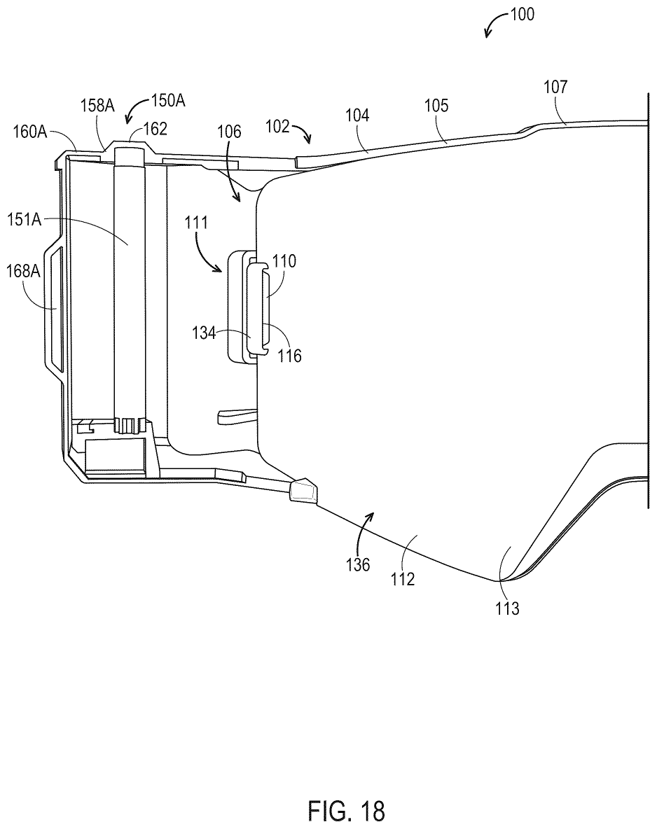

FIGS. 18-20 illustrate latch mechanism 111 of goggle frame 102. Goggle frame 102 may include any suitable latch mechanism 111 that is configured to be transitioned between a locked position and an unlocked position to selectively engage and disengage the second lateral end of lens 112. When latch mechanism 111 is in the locked position, latch mechanism is configured to secure the second lateral end of lens 112 to goggle frame 102, and when latch mechanism 111 is in the unlocked position, the second lateral end of lens 112 is permitted to be inserted in or removed from lens receiving area 106.

As shown in FIGS. 18-20, in some examples, latch mechanism 111 includes a latch locking member 110 and a latch guide 118. Latch locking member 110 is configured to be selectively transitioned between a locked position and an unlocked position. Latch locking member 110 is configured to operatively mate with latch-receiving aperture 116 of lens 112 when latch locking member 110 is in the locked position and lens 112 is operatively received in the lens receiving area 106, e.g., when lens 112 is pivoted about pin 108, such that the second lateral end of lens 112 is engaged with first frame segment 104 and disposed in lens receiving area 106. Latch locking member 110 is configured to retain the second lateral end of lens 112 in lens receiving area 106 when latch locking member 110 is in the locked position and mated with latch-receiving aperture 116. Latch locking member 110 is configured to be disengaged from lens 112 when latch locking member 110 is in the unlocked position. The second lateral end of lens 112 is permitted to moved relative to lens receiving area 106 when latch locking member 110 is in the unlocked for position. For example, the second lateral end of lens 112 may be received in and removed from lens receiving area 106 when latch locking member 110 is in the unlocked position.

As discussed above, latch-receiving aperture 116 of lens 112 is formed in or by lens tab 134 (AKA integral lens tab) that projects forward from the front surface of main-body portion 113 of lens 112 at or proximate the second lateral end of lens 112. As such, latch locking member 110 is configured to cover and/or engage the front surface of main-body portion 113 of lens 112 when latch locking member 110 is disposed in the locked position and is received in latch-receiving aperture 116. Explained in other words, latch locking member 110 is configured to be disposed between lens tab 134 and the front surface of main-body portion 113 of lens 112 when latch locking member 110 is mated with latch-receiving aperture 116.

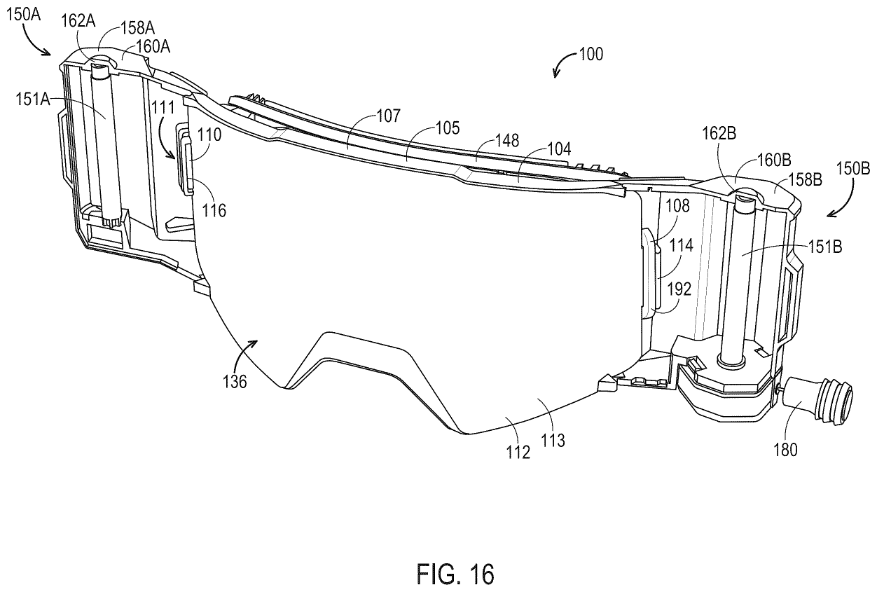

In some examples, latch locking member 110 is configured to be selectively translated relative to goggle frame 102 between the locked and unlocked positions. For example, as shown most clearly in FIG. 3, an end of latch locking member 110 is accessible on a rear side of first frame segment 104 and the end of latch locking member 110 is configured to be held by a user to permit the user to translate latch locking member 110 between the locked and unlocked positions. In some examples, translating latch locking member 110 in a first direction (e.g., outward) translates latch locking member 110 from the locked position to the unlocked position and translating latch locking member 110 in a second direction (e.g., inward) opposite the first direction translates latch locking member 110 from the unlocked position to the locked position. In FIGS. 16-20, latch locking member 110 is illustrated in the locked position, in which latch locking member 110 is mated with latch-receiving aperture 116 of lens 112.

Latch locking member 110 may comprise any suitable structures configured to be selectively engaged with the second lateral end of lens 112 (e.g., received in latch-receiving aperture 116) and configured to retain the second lateral end of lens 112 in lens receiving area 106, e.g., retain the second lateral end of lens 112 in a fixed position relative to first frame segment 104. For example, latch locking member 110 may comprise any suitable material having suitable rigidity to prevent the second lateral end of lens 112 from being moved relative first frame segment 104 when latch locking member 110 is engaged with the second lateral end of lens 112 and mated with latch-receiving aperture 116 of lens 112.

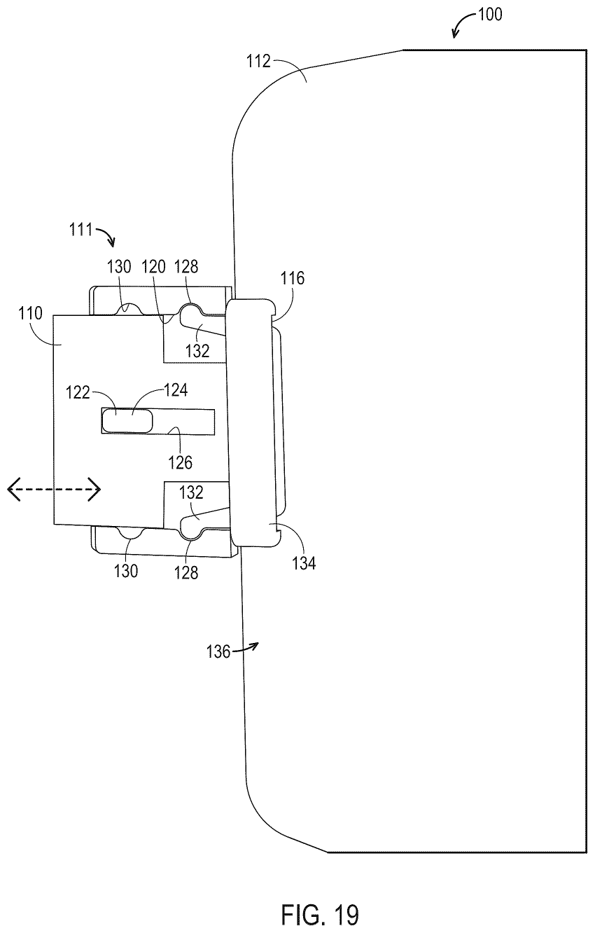

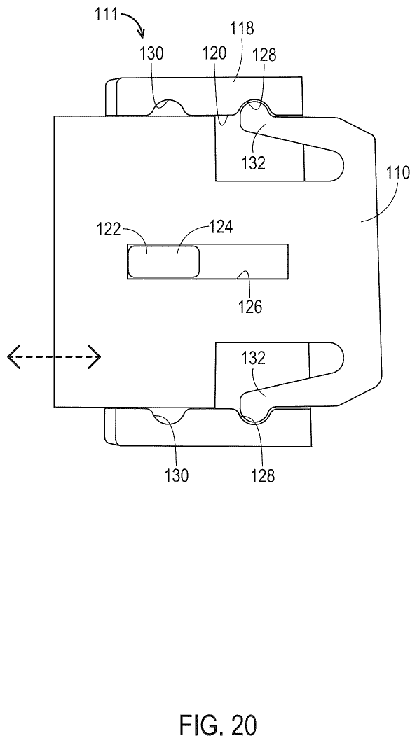

As shown in FIGS. 19 and 20, latch mechanism 111 includes a latch guide 118 configured to guide and limit the translation of latch locking member 110 relative to goggle frame 102 between the locked and unlocked positions. Latch guide 118 includes or defines a guide channel 120 (AKA guide groove) and latch locking member 110 is operatively received (e.g., slidingly received) in guide channel 120, such that latch locking member 110 is configured to be selectively translated in guide channel 120 between the locked and unlocked positions. For example, latch locking member 110 is configured to slide in guide channel 120 between the locked and unlocked positions. Latch guide 118 may be coupled or fastened to goggle frame 102 (e.g., first frame segment 104) in any suitable manner configured to maintain latch guide 118 in a fixed position relative to first frame segment 104. For example, latch guide 118 may be fixed to goggle frame 102 (e.g., first frame segment 104) using single-use fastener(s) (e.g., bolts, screws, etc.) and/or latch guide 118 and/or goggle frame 102 may include one or more detents and/or any other suitable engagement features configured to hold latch guide 118 in a fixed position relative to goggle frame 102. In some examples, first frame segment 104 includes a recess or cavity that is configured to receive and house latch mechanism 111 including latch guide 118 and latch locking member 110.

Latch guide 118 includes a retainment feature 122 (e.g., an abutment) disposed within guide channel 120 configured to limit the translation of latch locking member 110 in guide channel 120 and/or configured to retain latch locking member 110 in guide channel 120. In some examples, retainment feature 122 comprises a retainment protrusion 124 that is operatively received in an elongate opening 126 formed in latch locking member 110. Retainment protrusion 124 and elongate opening 126 are configured to prevent latch locking member 110 from being translated beyond the locked and unlocked positions and prevent latch locking member 110 from being removed from guide channel 120 during operation.

In some examples, latch guide 118 and/or latch locking member 110 include one or more structures and/or features that are configured to engage each other to releasably retain latch locking member 110 in the locked position and/or the unlocked position within guide channel 120. For example, as shown in FIGS. 19 and 20, latch guide 118 may include a pair of locked-arm notches 128 and a pair of unlocked-arm notches 130 each formed in guide channel 120. Latch locking member 110 includes a pair of flexible arms 132 that are configured to be operatively received in the locked-arm notches 128 when latch locking member 110 is in the locked position and configured to be operatively received in the unlocked-arm notches 130 when latch locking member 110 is in the unlocked position. Flexible arms 132 are configured to selectively flex or bend inward, such that flexible arms 132 are automatically released from the locked-arm notches 128 or the unlocked-arm notches 130 when latch locking member 110 is translated out of the locked position or unlocked position by the user. Flexible arms 132 automatically return to an unflexed position when flexible arms 132 are aligned with locked-arm notches 128 or unlocked-arm notches 130, such that flexible arms 132 are automatically received in the locked-arm notches 128 or unlocked-arm notches 130. Explained in other words, locked-arm notches 128 and unlocked-arm notches 130 are configured to releasably retain flexible arms 132 to prevent undesired translation of latch locking member 110 out of the locked and unlocked positions during use. When the user translates latch locking member 110 out of the locked or unlocked position, flexible arms 132 are configured to flex or bend inward, such that flexible arms 132 are automatically removed or released from the locked-arm or unlocked-arm notches 130, 130 and latch locking member 110 is permitted to be translated between the locked and unlocked positions as desired by the user.

In FIGS. 19 and 20, flexible arms 132 are disposed in an unflexed or outward position and flexible arms 132 are operatively received in locked-arm notches 128 of latch guide 118. Flexible arms 132 of latch locking member 110 are configured to flex into a flexed or inward position when latch locking member 110 is translated from the locked position to the unlocked position in guide channel 120, such that flexible arms 132 are automatically released from locked-arm notches 128. When flexible arms 132 align with unlocked-arm notches 130, flexible arms 132 return to the unflexed or outward position, such that flexible arms 132 are operatively received in unlocked-arm notches 130.

In general, lens 112 is operatively retained and secured in lens receiving area 106 when pin 108 is operatively engaged with the hook 114 and the latch locking member 110 is operatively mated with latch-receiving aperture 116. Lens 112 is permitted to be selectively removed from lens receiving area 106 by translating latch locking member 110 into the unlocked position, such that latch locking member 110 is removed from latch-receiving aperture 116 and disengaged from lens 112. Lens 112 may then be pivoted about pin 108 to remove the second lateral end of lens 112 from lens receiving area 106 and hook 114 may be disengaged from pin 108, such that lens 112 is disengaged from goggle frame 102.

In the example of goggle 100 shown in FIGS. 2-24, integral hook 114 is disposed at a left lateral end of lens 112 and latch-receiving aperture 116 is disposed at the right lateral end of lens 112. Accordingly, pin 108 is disposed at a left lateral end of lens receiving area 106 and latch mechanism 111 is disposed at a right lateral end of lens receiving area 106. In such examples, the left lateral end of lens 112 is configured to be pivotably coupled to first frame segment 104 using integral hook 114 and pin 108 and the right lateral end of lens 112 is configured to be latched to first frame segment 104 using latch mechanism 111. Alternatively, in some examples, integral hook 114 may be disposed at the right lateral end of lens 112, latch-receiving aperture 116 may be disposed at the left lateral end of lens 112, pin 108 may be disposed at the right lateral end of lens receiving area 106, and latch mechanism 111 may be disposed at the left lateral end of lens receiving area 106.

As shown in FIGS. 21-24, first and second canisters 150A, 150B each include a respective canister cover 164A, 164B configured to close the respective canister 150A, 150B and contain supply roll 152 or take-up roll 156 within the respective canister 150A, 150B during operation. Each canister cover 164A, 164B is configured to be selectively and operatively coupled to the integral part 158A, 158B of first frame segment 104 that forms the respective portion (e.g., rear portion) of canisters 150A, 150B.

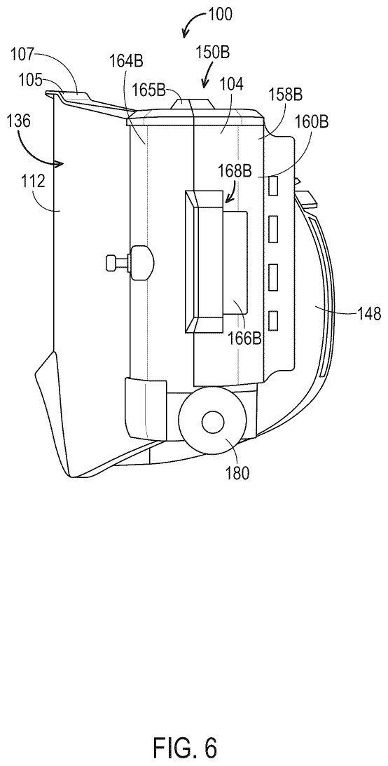

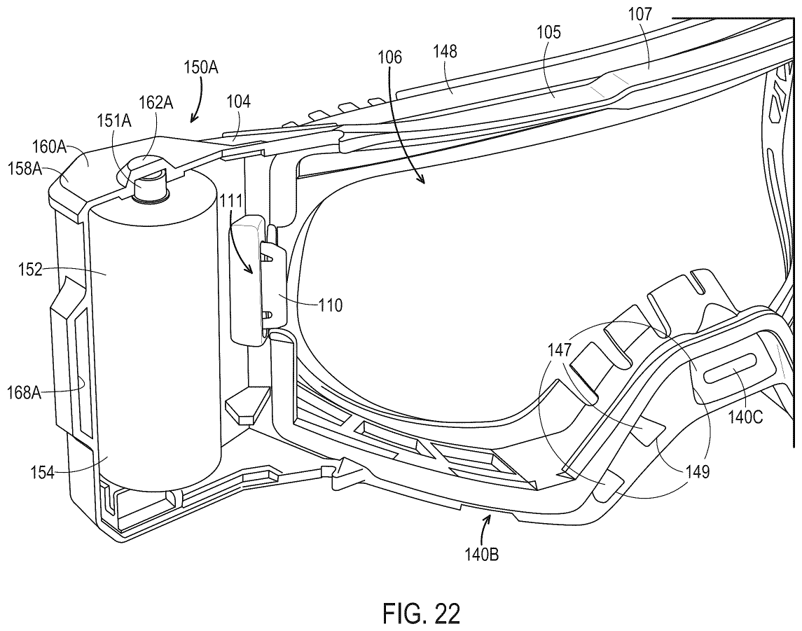

First and second canister covers 164A, 164B are each configured to be operatively coupled to integral parts 158A, 158B of first frame segment in any suitable manner, such that first and second canister covers 164A, 164B are configured to be removed and replaced repeatedly and manually without damage. In some examples, as shown in FIGS. 21 and 22, first integral part 158A of first frame segment 104 that forms the rear portion of first canister 150A includes a first tab opening 168A and first canister cover 164A includes a first locking tab 166A configured to operatively mate with first tab opening 168A to releasably couple first canister cover 164A to first integral part 158A of first frame segment 104 to form first canister 150A. First canister 150A is configured to operatively support and house supply roll 152 of lens film 154. As shown most clearly in FIG. 6, second integral part 158B of first frame segment 104 includes a substantially similar second tab opening 168B and second canister cover 164B includes a substantially similar second locking tab 166B configured to releasably couple second canister cover 164B to second integral part 158B to form second canister 150B. Second canister 150B is configured to operatively support and house take-up roll 156 of lens film 154. As discussed above, goggle frame 102 may include any suitable mechanism (e.g., pull-cord ratchet mechanism 180) that is operatively coupled to second canister 150B and configured to be actuated by the user to rotate take-up roll 156 to selectively index lens film 154 across the front surface of lens 112 from supply roll 152 to take-up roll 156.

In some examples, the attachment mechanism(s) for coupling lens 112 to goggle frame 102 (e.g., latch mechanism 111 and/or pin 108) are disposed within first and second canisters 150A, 150B when goggle 100 is assembled. For example, as shown in FIGS. 21-22, canister covers 164A, 164B are each configured to cover or extend in front of a portion of lens receiving area 106 when canister covers 164A, 164B are operatively coupled to respective integral parts 158A, 158B of first frame segment 104. In some examples, first canister cover 164A of first canister 150A is configured to cover at least a portion of the second lateral end of lens receiving area 106 at which latch mechanism 111 is disposed and second canister cover 164B of second canister 150B is configured to cover at least a portion of the first lateral end of lens receiving area 106 at which pin 108 is disposed. In such examples, canister covers 164A, 164B may be configured to extend in front of the lateral ends of lens 112 when lens 112 is operatively received in lens receiving area 106. For example, first canister cover 164A may be configured to cover latch-receiving aperture 116 of lens 112 that is operatively mated with latch locking member 110 of goggle frame 102 and second canister cover 164B may be configured to cover integral hook 114 of lens 112 operatively engaged with pin 108 of goggle frame 102. In such examples, canister covers 164A, 164B may be spaced from the front surface of the lateral ends of lens 112 to form respective film gaps through which lens film 154 is permitted to extend from supply roll 152 within first canister 150A to take-up roll 156 within second canister 150B across the front surface of lens 112.

In some examples, canister covers 164A, 164B are configured to be installed on goggle frame (e.g., releasably coupled to integral parts 158A, 158B) after lens 112 has been operatively coupled to goggle frame. For example, because canister covers 164A, 164B are configured to cover the lateral ends of lens 112 and lens receiving area 106, assembling goggle 100 may include releasably coupling canister covers 164A, 164B to first frame segment 104 after coupling lens 112 to goggle frame and after operatively inserting supply roll 152 and take-up roll 156 of lens film 154 into canisters 150A, 150B.

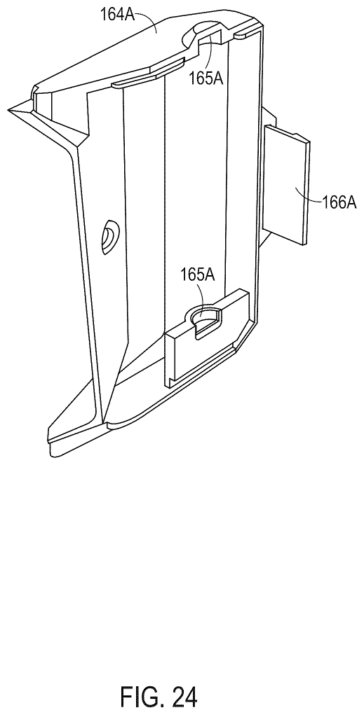

As shown in FIG. 24, in some examples, first canister cover 164A of first canister 150A includes a respective spindle holder portion 165A configured to form a portion of a spindle holder for supply roll 152. For example, when first canister cover 164A is coupled to first integral part 158A of first frame segment 104, first integral part 158A of first frame segment 104 and spindle holder portion 165A of first canister cover 164A collectively form a supply-roll spindle holder 162A configured to removably retain a supply-roll spindle 151A on which supply roll 152 is mounted. In some examples, second canister cover 164A includes a respective spindle holder portion 165B similar to spindle holder portion 165A of first canister cover 164B shown in FIG. 24, such that second canister cover 164A and second integral part 158B of first frame segment 104 collectively form a take-up roll spindle holder 162B of second canister 150B.

In the example of goggle 100 shown in FIGS. 2-24, first canister 150A disposed at the right lateral side of goggle 100 is configured to house supply roll 152 and second canister 150B disposed at the left lateral side of goggle 100 is configured to house take-up roll 156 of lens film 154. In this example, lens film 154 is configured to be conveyed right to left across the front surface of lens 112. Alternatively, in some examples, goggle 100 may include a supply canister housing a supply roll 152 that is disposed at the left lateral side of goggle 100 and a take-up canister housing a take-up roll at the right lateral side of goggle 100.

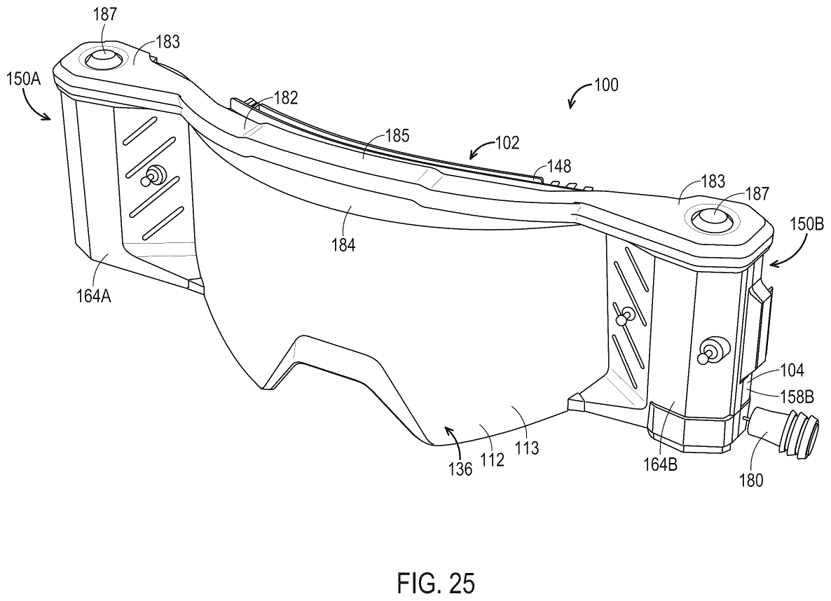

FIGS. 25-29 illustrate an example mud visor 182 for use with goggle 100. Mud visor 182 is configured to be releasably coupled to the goggle frame 102 during use. For example, mud visor 182 may comprise an elastic material (e.g., Thermoplastic Polyurethane (TPU)) configured to deform (e.g., stretch) to permit releasably coupling mud visor 182 to goggle frame 102 (e.g., first frame segment 104) in a press-fit or friction-fit arrangement. For example, mud visor 182 is configured to elastically deform when pressed onto the upper surface of goggle frame 102 to create a frictional or interference fit between the elastic body of mud visor 182 and the upper surfaces of goggle frame 102.

In some examples, mud visor 182 is configured to be coupled to an upper side of first frame segment 104, such that mud visor 182 extends over an upper surface of first frame segment 104, first and second integral parts 158A, 158B of first frame segment 104 that form the rear portions of first and second integral canisters 150A, 150B, and/or first and second canister covers 164A, 164B of first and second canisters 150A, 150B. Explained in other words, mud visor 182 is configured to cover an upper surface of first frame segment 104 and first and second integral canisters 150A, 150B including canister covers 164A, 164B. In some examples, mud visor 182 is configured to be installed on goggle 100 after lens 112 and first and second canister covers 164A, 164B have been operatively coupled to first frame segment 104.

In some examples, mud visor 182 has a shape, size, and/or profile that is configured to generally match a shape, size, and/or profile of goggle frame 102 and/or lens 112. For example, as shown in FIGS. 25-29, lateral end portions 183 of mud visor 182 are sized and shaped to extend over and couple to first and second integral canisters 150A, 150B and a central portion 185 of mud visor 182 includes a raised portion configured to accommodate raised central portion 174 of lens 112 and a frame raised central portion 175 of goggle frame 102. In some examples, lateral end portions 183 of mud visor 182 include raised-spindle portions 187 configured to accommodate or mate with raised portions of first and second integral canisters 150A, 150B formed by external surfaces of spindle holders 162A, 162B. In some examples, mud visor 182 comprises a monolithic, single-piece structure. For example, central portion 185, lateral end portions 183, and a mud flap 184 of mud visor 182 are formed as a single piece structure.

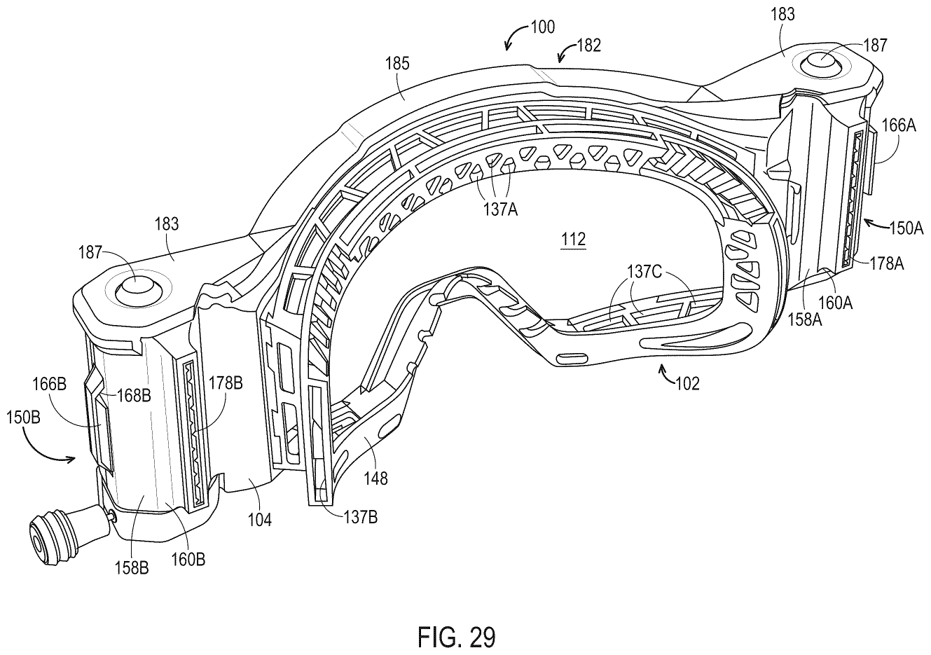

In some examples, mud visor 182 is configured to assist with securing canisters 150A, 150B in a closed position during use. For example, as shown in FIGS. 25 and 29, lateral end portions 183 of mud visor 182 are configured to extend over and to cover an entirety of the upper sides of first and second canisters 150A, 150B including the rear portions of canisters 150A, 150B that are formed by integral parts 158A, 158B of first frame segment 104 and canister covers 164A, 164B. In some examples, mud visor 182 comprises a resilient material that is configured to deform elastically (e.g., stretch) to cover the entirety of the upper side of integral canisters 150A, 150B and that is configured to apply an elastic force that assists with securing canister covers 164A, 164B to integral parts 158A, 158B. As such, mud visor 182 may be configured to prevent undesired decoupling of canister covers 164A, 164B from integral parts 158A, 158B of first frame segment 104 during use.

Mud visor 182 comprises a mud flap 184 that extends downward from mud visor 182. Mud flap 184 is configured to extend in front of an upper portion of lens 112 when mud visor 182 is operatively coupled to the goggle frame 102. When lens film 154 extends across the front surface of lens 112, mud flap 184 is configured to extend in front of and cover an upper edge of lens film 154 to prevent dirt and debris from entering between lens film 154 and the front surface of lens 112. For example, mud flap 184 is configured to prevent dirt and debris from entering between the front surface of lens 112 and lens film 154 at the upper edge of lens film 154. Mud flap 184 may be configured to extend downward to cover any suitable portion of lens 112, such that mud flap 184 extends in front of and covers the upper edge of lens film 154 without obstructing the user's view through lens 112. Mud flap 184 may be spaced in front of the front surface of lens 112 and lens film 154 or mud flap 184 may be configured to directly contact lens film 154 and/or the front surface of lens 112 when installed on goggle frame 102.

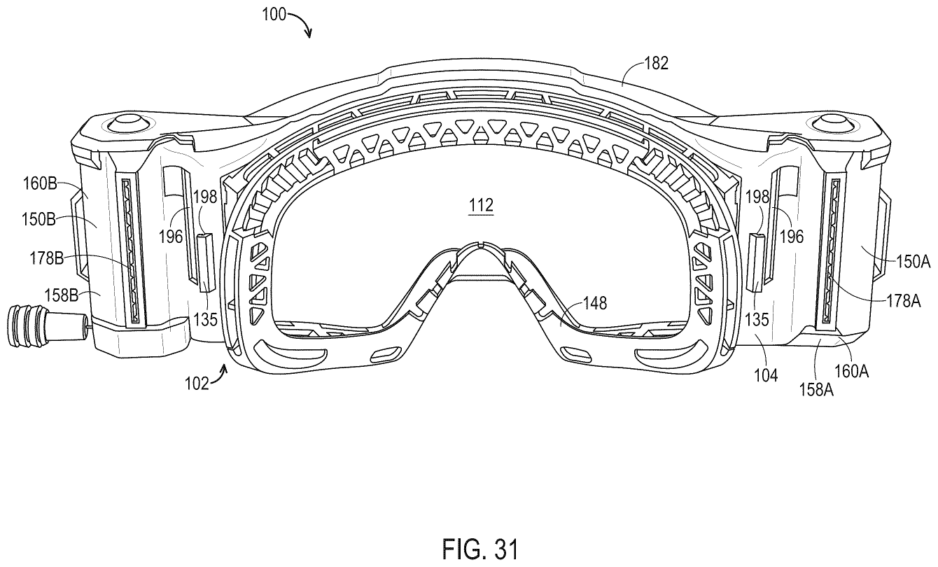

FIGS. 30-33 show an illustrative example of goggle 100 discussed above with reference to FIGS. 1-29 that includes a second illustrative lens attachment system. As shown in FIGS. 30-31, in some examples, goggle 100 includes a pair of latch mechanisms 111 each comprising a latch locking member 110 configured to operatively secure lens 112 to first frame segment 104 and goggle frame 102. Explained in other words, rather than lens 112 being pivotably coupled to goggle frame 102 at a first lateral end and being latched to goggle frame 102 at a second lateral end (e.g., as shown in the examples of FIGS. 1-29), goggle frame 102 may include a pair of latch mechanisms 111 configured to releasably latch both first and second lateral ends of lens 112 to goggle frame 102.

In some examples, each latch mechanism 111 is operatively coupled to and supported by a respective one of the integral parts of goggle frame 102 that form at least a respective portion of integral film canisters 150A, 150B. Explained in other words, each latch mechanism 111 may be supported by a respective one of integral film canisters 150A, 150B of goggle frame 102. For example, as shown in FIGS. 30-31, each latch mechanism 111 is operatively coupled to a rear side of integral parts 158A, 158B of first frame segment 104 that form respective rear portions 160A, 160B of integral film canisters 150A, 150B.

Each latch mechanism 111 may include any suitable structure(s) that are configured to selectively and releasably secure lens 112 to goggle frame 102. For example, each latch mechanism 111 may include a latch locking member 110 that is configured to be selectively transitioned (e.g., moved) between a locked position (as shown in FIG. 30) and an unlocked position. When in the locked position, latch locking member 110 is engaged with a portion of lens 112 to secure lens 112 to goggle frame 102. When in the unlocked position, latch locking member 110 is disengaged from lens 112 and lens 112 is removable from goggle frame 102. In some examples, latch locking members 110 of latch mechanisms 111 are configured to be selectively transitioned between the locked and unlocked positions independently of each other.

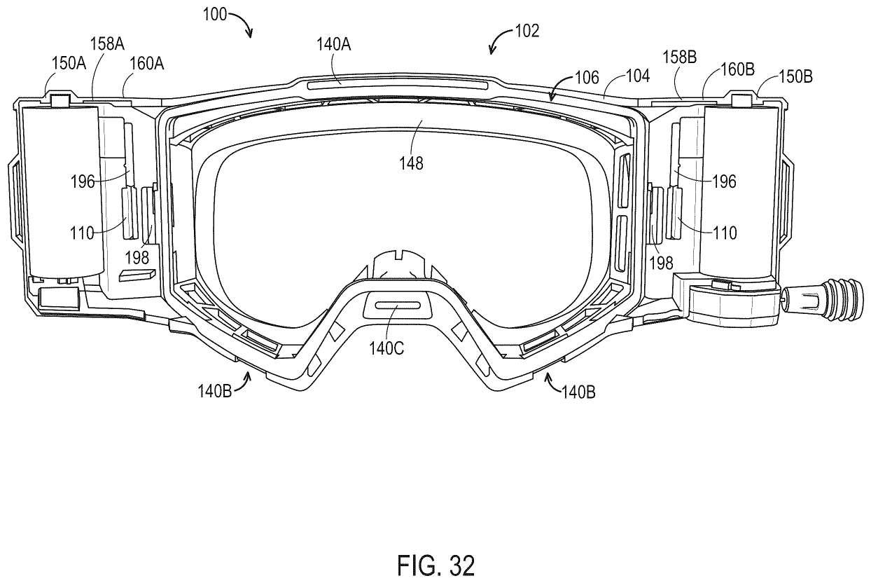

Latch locking members 110 may be configured to be selectively transitioned (e.g., moved) between the locked and unlocked positions in any suitable manner. For example, as shown in FIGS. 30-33, each latch locking member 110 is slidingly coupled to the rear side of first frame segment 104, such that each latch locking member 110 is configured to be selectively transitioned between the locked and unlocked positions by sliding latch locking members 110 relative to first frame segment 104. As shown in FIG. 31, a pair of latch slots 196 are formed in first frame segment 104 and each latch locking member 110 is operatively coupled to a respective one of latch slots 196. Latch slots 196 permit selectively sliding each latch locking member 110 relative to first frame segment 104 between the locked and unlocked positions. In some examples, as shown in FIG. 32, a portion of each of latch locking members 110 is configured to be slidingly received in a respective one of latch slots 196, such that latch locking members 110 are slidingly coupled to first frame segment 104.

As shown in FIG. 31, in some examples, each latch slot 196 extends substantially vertically on the rear side of first frame segment 104, such that latch locking members 110 are configured to slide substantially vertically between the locked and unlocked positions. For example, sliding latch locking member 110 upward transitions latch locking member 110 into the unlocked position and sliding latch locking member 110 downward transitions latch locking member 110 into the locked position.

In some examples, each latch locking member 110 may be biased into the locked position, e.g., by a spring. For example, a spring and/or any other suitable biasing mechanism may be configured to maintain latch locking member 110 in the locked position unless a user applies a force to transition latch locking member 110 from the locked position to the unlocked position. In such examples, the spring and/or other suitable biasing mechanism may automatically transition latch locking member 110 into the locked position after latch locking member 110 is released by the user. Alternatively, latch locking member 110 may not be biased into the locked position or the unlocked position and latch locking member 110 may be configured to be manually transitioned between the locked and unlocked positions by the user.

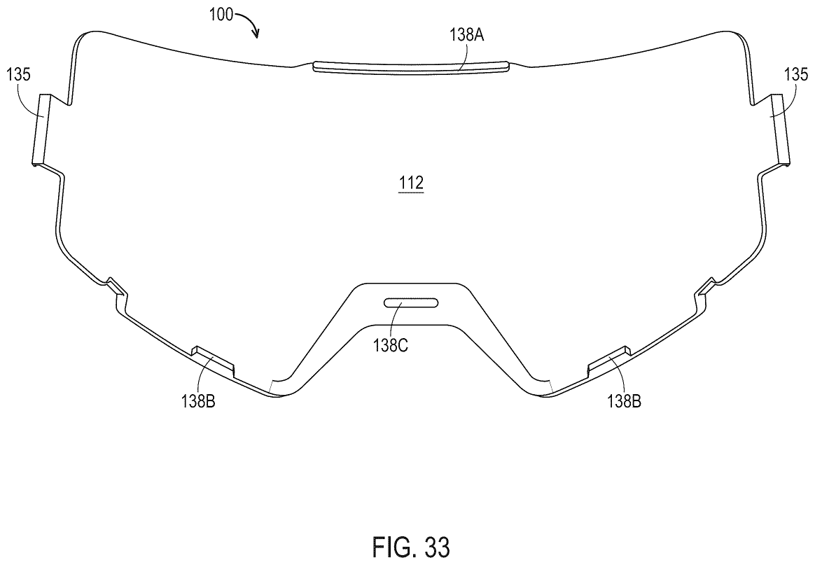

In some examples, lens 112 may include any suitable features configured to be selectively engaged by the pair of latch locking members 110. As shown most clearly in FIGS. 31 and 33, in some examples, lens 112 includes a pair of latch-engagement tabs 135 that project laterally outward from first and second lateral ends of lens 112. Each latch-engagement tab 135 is configured to be selectively engaged by a respective one of latch locking members 110 to secure lens 112 to goggle frame 102. Latch-engagement tabs 135 may have any suitable shape and/or size to facilitate being securely held by a respective one of latch locking members 110. For example, as shown in FIGS. 31 and 33, latch-engagement tabs 135 may comprise an L-shaped and/or right-angled tab having a hooked-end portion that is configured to facilitate latch locking members 110 securely holding latch-engagement tabs 135. In some examples, latch-engagement tabs 135 are integral with lens 112, e.g., lens 112 comprises a single-piece monolithic structure including latch-engagement tabs 135.

As shown in FIGS. 31 and 32, in some examples, first frame segment 104 includes a pair of tab-receiving apertures 198 each extending through first frame segment 104 from a front side to a rear side of first frame segment 104. Each tab-receiving aperture 198 is configured to selectively receive a respective one of latch-engagement tabs 135 when lens 112 is received in lens receiving area 106. In some examples, latch-engagement tabs 135 are configured to extend through tab-receiving apertures 198, such that latch-engagement tabs 135 project outward from the rear side of first frame segment 104. This positions latch-engagement tabs 135 on the rear side of first frame segment 104 and in a suitable position to be selectively engaged by latch locking members 110, which are disposed on the rear side of first frame segment 104.

In some examples, as shown in FIG. 32, tab-receiving apertures 198 are disposed on opposing lateral ends of lens receiving area 106, such that tab-receiving apertures 198 are positioned to receive latch-engagement tabs 135 disposed at opposing lateral ends of lens 112, e.g., when lens 112 is operatively received in lens receiving area 106. For example, tab-receiving apertures 198 may be formed in left and right integral parts 158A, 158B of first frame segment 104 that form the respective portions of left and right integral film canisters 150A, 150B of goggle frame 102. In some examples, goggle 100 includes a pair of canister covers 164A, 164B that are configured to cover the front side of first and second lateral ends of lens 112 and lens receiving area 106 when goggle 100 is assembled.

### B. Illustrative Method

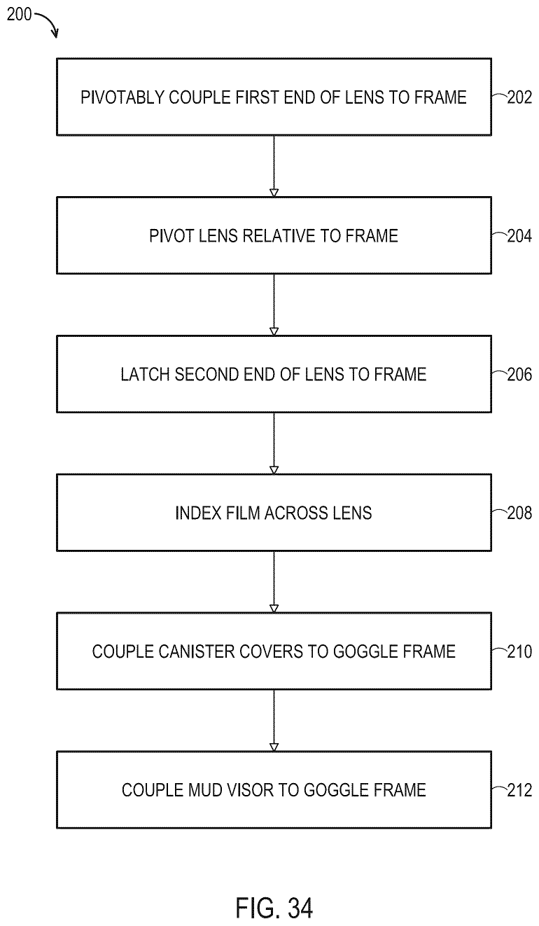

This section describes steps of an illustrative method 200 for assembling a protective goggle, such as goggle 100 discussed above; see FIG. 34. Aspects of goggle 100 discussed above may be utilized in the method steps described below. Where appropriate, reference may be made to components and systems that may be used in carrying out each step. These references are for illustration, and are not intended to limit the possible ways of carrying out any particular step of the method.

FIG. 34 is a flowchart illustrating steps performed in an illustrative method, and may not recite the complete process or all steps of the method. Although various steps of method 200 are described below and depicted in FIG. 34, the steps need not necessarily all be performed, and in some cases may be performed simultaneously or in a different order than the order shown.

Step 202 of method 200 includes pivotably coupling a first lateral end of a lens to a goggle frame. For example, as discussed above with reference to goggle 100, lens 112 may comprise an integral hook 114 disposed at a first lateral end of lens 112 and goggle frame 102 includes a pin 108 disposed at a first lateral end of a lens receiving area 106 of goggle frame 102. In such examples, pivotably coupling the first lateral end of lens 112 to goggle frame 102 in step 202 includes operatively engaging integral hook 114 with pin 108.

Step 204 of method 200 includes pivoting the lens relative to the goggle frame, such that a second lateral end of the lens is received in a lens receiving area of the goggle frame. For example, when integral hook 114 is operatively engaged with pin 108 in step 202, lens 112 is selectively pivotable about pin 108 relative to goggle frame 102. In step 204, lens 112 is pivoted about pin 108 to position the second lateral end of lens 112 in contact with goggle frame 102 (e.g., first frame segment 104) and in a lens receiving area 106 formed by goggle frame 102.

Step 206 of method 200 includes latching the second lateral end of the lens to the goggle frame to secure the lens to the goggle frame, e.g., in the lens receiving area of the goggle frame. In some examples, goggle frame 102 comprises a latch mechanism 111 including a latch locking member 110. In such examples, latching the second lateral end of lens 112 to goggle frame 102 includes transitioning latch locking member 110 of latch mechanism 111 into a locked position, in which latch locking member 110 operatively engages the second lateral end of lens 112 to secure and retain the second lateral end of lens 112 in lens receiving area 106. In some examples, the second lateral end of lens 112 comprises a latch-receiving aperture 116 and latch locking member 110 is operatively mated with latch-receiving aperture 116 when latch locking member 110 is transitioned into the locked position. In some examples, latch-receiving aperture 116 is formed by an integral tab 134 projecting forward from a front surface of a main-body portion 113 of lens 112, such that latch-receiving aperture 116 is disposed in front of the front surface of main-body portion 113 of lens 112. In such examples, latch locking member 110 is configured to cover and/or engage the front surface of main-body portion 113 when latch locking member 110 is operatively mated (e.g., received in) with latch-receiving aperture 116. For example, latch locking member 110 is configured to be selectively received between integral tab 134 and the front surface of main-body portion 113 within latch-receiving aperture 116.

In some examples, rather than performing steps 202-206, method 200 includes latching each of the first and second lateral end of lens 112 to goggle frame 102. For example, as shown in FIGS. 30-33, goggle 100 may include a pair of latch mechanisms 111 each including a latch locking member 110 that is configured to selectively and releasably engage a respective lateral portion of lens 112 (e.g., a latch-engagement tab 135) to secure lens 112 to goggle frame 102. In such examples, method 200 may include selectively transitioning each latch locking member 110 of the pair of latch mechanisms 111 into a locked position, such that the pair of latch locking members 110 engage the first and second lateral ends of lens 112 to secure lens 112 to goggle frame 102.

In some examples, method 200 includes a step 208 comprising indexing a lens film 154 across a front surface of lens 112 from a supply roll 152 of lens film 154 to a take-up roll 156. In some examples, supply roll 152 is disposed in a first integral canister 150A of goggle frame 102 and take-up roll 156 is disposed in a second integral canister 150B of goggle frame 102 and the first and second integral canisters are disposed on opposing lateral sides of lens receiving area 106. In some examples, indexing lens film 154 across the front surface of lens 112 includes operatively inserting a supply roll 152 of lens film 154 into first canister 150A, extending lens film 154 from supply roll 152 across the front surface of lens 112 and operatively coupling lens film 154 to a take-up spindle 151B of second canister 150B. In such examples, rotating take-up spindle 151B (e.g., using pull-cord ratchet mechanism 180) pulls lens film 154 from supply roll 152 to take-up roll 156 to add the used film to take-up roll 156.

In some examples, method 200 includes a step 210 comprising releasably coupling canister covers 164A, 164B to first frame segment 104 to form first and second film canisters 150A, 150B of goggle frame 102. Canister covers 164A, 164B are configured to enclose and contain supply roll 152 and take-up roll 156 of lens film 154 within first and second canisters 150A, 150B. In some examples, canister covers 164A, 164B are configured to extend in front of and cover lateral end portions of lens 112 and/or lens receiving area 106.

In some examples, method 200 includes a step 210 comprising releasably coupling a mud visor 182 to an upper portion of goggle frame 102, such that a mud flap 184 of mud visor 182 extends downward in front of an upper portion of lens 112. Mud flap 184 of mud visor 182 is configured to extend in front of an upper edge of lens film 154 extending across the front surface of lens 112 to prevent dirt and debris from entering between lens 112 and lens film 154 at the upper edge of lens film 154.

### C. Illustrative Combinations and Additional Examples

This section describes additional aspects and features of protective goggles, presented without limitation as a series of paragraphs, some or all of which may be alphanumerically designated for clarity and efficiency. Each of these paragraphs can be combined with one or more other paragraphs, and/or with disclosure from elsewhere in this application, including the materials incorporated by reference in the Cross-References, in any suitable manner. Some of the paragraphs below expressly refer to and further limit other paragraphs, providing without limitation examples of some of the suitable combinations.
A0. A protective goggle (100), comprising:
a goggle frame (102) including a first frame segment (104) forming a lens receiving area (106), wherein the goggle frame (102) comprises a pin (108) disposed at a first lateral end of the lens receiving area (106) and a latch locking member (110) disposed at a second lateral end of the lens receiving area (106); and
a lens (112) configured to be operatively received in the lens receiving area (106), wherein the lens (112) comprises a hook (114) disposed at a first lateral end of the lens (112) and a latch-receiving aperture (116) disposed at a second lateral end of the lens (112);
wherein the pin (108) is configured to operatively engage the hook (114) and the latch locking member (110) is configured to operatively mate with the latch-receiving aperture (116) to secure the lens (112) to the goggle frame (102).
A1. The goggle (100) of paragraph A0, wherein the lens (112) is configured to be pivoted about the pin (108) when the hook (114) is operatively engaged with the pin (108) and the latch locking member (110) is disengaged from the lens (112).
A2. The goggle (100) of paragraph A0 or A1, wherein the latch locking member (110) is configured to be selectively transitioned between a locked position and an unlocked position.
A2.1. The goggle (100) of paragraph A2, wherein the latch locking member (110) is configured to operatively mate with the latch-receiving aperture (116) when the latch locking member (110) is in the locked position and the lens (112) is operatively received in the lens receiving area (106), and wherein the latch locking member (110) is configured to be disengaged from the lens (112) when the latch locking member (110) is in the unlocked position.
A2.2. The goggle (100) of paragraph A2 or A2.1, wherein the latch locking member (110) is configured to operatively retain the lens (112) in the lens receiving area (106) when the latch locking member (110) is in the locked position and is operatively mated with the latch-receiving aperture (116).
A2.3. The goggle (100) of any one of paragraphs A2-A2.2, wherein the lens (112) is permitted to be selectively and operatively received in and removed from the lens receiving area (106) when the latch locking member (110) is in the unlocked position.
A2.4. The goggle (100) of any one of paragraphs A2-A2.3, wherein the latch locking member (110) is configured to be selectively translated relative to the goggle frame (102) between the locked position and the unlocked position.
A2.5. The goggle (100) of paragraph A2.4, wherein an end of the latch locking member (110) is accessible on a rear side of the first frame segment (104).
A2.6. The goggle (100) of paragraph A2.5, wherein the end of the latch locking member (110) is configured to be held by a user to permit the user to translate the latch locking member (110) between the locked and unlocked positions.
A2.7. The goggle (100) of any one of paragraphs A2.4-A2.7, wherein translating the latch locking member (110) in a first direction translates the latch locking member (110) from the locked position to the unlocked position, and wherein translating the latch locking member (110) in a second direction opposite the first direction translates the latch locking member (110) from the unlocked position to the locked position.
A2.8. The goggle (100) of any one of paragraphs A2-A2.7, further comprising a latch guide (118) including a guide channel (120), wherein the latch locking member (110) is operatively received in the guide channel (120), such that the latch locking member (110) is configured to be selectively translated in the guide channel (120) between the locked and unlocked positions.
A2.9. The goggle (100) of paragraph A2.8, wherein the latch guide (118) is operatively fixed to the goggle frame (102), e.g., to the first frame segment (104).
A2.10. The goggle (100) of paragraph A2.8 or A2.9, wherein the latch guide (118) includes a retainment feature (122) configured to limit translation of the latch locking member (110) in the guide channel (120) and configured to retain the latch locking member (110) in the guide channel (120).
A2.11. The goggle (100) of paragraph A2.10, wherein the retainment feature (122) comprises a retainment protrusion (124) operatively received in an elongate opening (126) formed in the latch locking member (110).
A2.12. The goggle (100) of paragraph A2.10 or A2.11, wherein the retainment feature (122) is configured to permit the latch locking member (110) to be translated between the locked and unlocked positions and prevent the latch locking member (110) from being removed from the guide channel (120) during operation.
A2.13. The goggle (100) of any one of paragraphs A2.8-A2.12, further comprising a pair of locked-arm notches (128) and a pair of unlocked-arm notches (130) formed in the guide channel (120), wherein the latch locking member (110) further comprises a pair of flexible arms (132) configured to be operatively received in the locked-arm notches (128) when the latch locking member (110) is in the locked position and configured to be operatively received in the unlocked-arm notches (130) when the latch locking member (110) is in the unlocked position.
A2.14. The goggle (100) of paragraph A2.13, wherein the locked-arm notches (128) and the unlocked-arm notches (130) are configured to releasably retain the flexible arms (132) to hold the latch locking member (110) in the locked and unlocked positions, respectively.
A2.15. The goggle (100) of paragraph A2.13 or A2.14, wherein the flexible arms (132) are configured to flex, such that the flexible arms (132) are automatically released from the locked-arm notches (128) or the unlocked-arm notches (130) when the latch locking member (110) is translated out of the locked position or unlocked position by the user.
A2.16. The goggle (100) of paragraph A2.13 or A2.15, wherein the flexible arms (132) are configured to automatically return to unflexed position when permitted, such that the flexible arms (132) are automatically received in the locked-arm notches (128) or the unlocked-arm notches (130) when the latch locking member (110) is translated into the locked position or unlocked position by the user.
A3. The goggle (100) of any one of the preceding paragraphs, wherein the lens (112) is operatively retained in the lens receiving area (106) when the pin (108) is operatively engaged with the hook (114) and the latch locking member (110) is operatively mated with the latch-receiving aperture (116).
A4. The goggle (100) of any one of the preceding paragraphs, wherein the hook (114) is integral with the first lateral end of the lens (112).
A5. The goggle (100) of any one of the preceding paragraphs, wherein the lens comprises an integral lens tab (134) and the latch-receiving aperture (116) is formed in the integral lens tab (134).
A5.1. The goggle (100) of paragraph A5, wherein the integral lens tab (134) projects forward from a front surface (136) of the lens (112) at or proximate the second lateral end of the lens (112).
A5.2. The goggle (100) of paragraph A5.1, wherein the latch locking member (110) is configured to engage the front surface (136) of the lens (112) when the latch locking member (110) is operatively received in the latch-receiving aperture (116).
A5.3. The goggle (100) of paragraph A5.1 or A5.2, wherein the latch locking member (110) is configured to cover a portion of the front surface (136) of the lens (112) when the latch locking member (110) is operatively received in the latch-receiving aperture (116).
A5.4. The goggle (100) of any one of paragraphs A5.1-A5.3, wherein the latch locking member (110) is disposed between the integral lens tab (134) and the front surface (136) of the lens (112) when the latch locking member (110) is operatively mated with the latch receiving aperture (116).
A5.5. The goggle (100) of any one of paragraphs A5-A5.4, wherein a body of the integral lens tab (134) defines the latch-receiving aperture (116).
A6. The goggle (100) of any one of the preceding paragraphs, wherein the latch-receiving aperture (116) is disposed forward of a front surface (136) of the lens (112), such that the latch locking member (110) is configured to engage a portion of the front surface (136) of the lens (112) when the latch locking member (110) is operatively mated with the latch-receiving aperture (116).
A6.1. The goggle (100) of paragraph A6, wherein the latch locking member (110) is configured to cover the portion of the front surface (136) of the lens (112) when operatively mated with the latch-receiving aperture (116).
A7. The goggle (100) of any one of the preceding paragraphs, wherein the first frame segment (104) forms a peripheral rim (105) defining the lens receiving area (106).
A7.1. The goggle (100) of paragraph A7, wherein an upper rim (107) of the peripheral rim (105) overhangs an upper edge of the lens (112) when the lens (112) is operatively received in the lens receiving area (106).
A7.2. The goggle (100) of paragraph A7 or A7.1, wherein the upper rim (107) includes a frame raised central portion (175) configured to accommodate a raised central portion (174) of the lens (112).
A8. The goggle (100) of any one of the preceding paragraphs, wherein the lens (112) comprising the hook (114) and a/the integral lens tab (134) is formed as a single piece.
A9. The goggle (100) of any one of the preceding paragraphs, wherein the lens (112) further comprises one or more engagement protrusions (138A-C) configured to operatively engage the goggle frame (102) when the lens (112) is operatively received in the lens receiving area (106).
A9.1. The goggle (100) of paragraph A9, wherein the goggle frame (102) further comprises one or more corresponding engagement pockets (140A-C) configured to receive the one or more engagement protrusions (138A-C) when the lens (112) is operatively received in the lens receiving area (106).
A9.2. The goggle (100) of paragraph A9 or A9.1, wherein the one or more engagement protrusions (138A-C) extend rearward from the lens (112), i.e., generally toward the user's face when the goggle (100) is worn by the user.
A9.3. The goggle (100) of any one of paragraphs A9-A9.2, wherein the one or more engagement protrusions (138A-C) include one or more of a top engagement protrusion (138A) disposed proximate a top edge of the lens (112), a pair of bottom engagement protrusions (138B) each disposed proximate a bottom edge of the lens (112), and/or a nose engagement protrusion (138C) disposed proximate a lower nose cutout (144) of the lens (112).
A9.4. The goggle (100) of paragraph A9.3, wherein the one or more corresponding engagement pockets (140A-C) include one or more of a top engagement pocket (140A) disposed proximate a top edge of the lens receiving area (106) and configured to receive the top engagement protrusion (138A), a pair of bottom engagement pockets (140B) each disposed proximate a bottom edge of the lens receiving area (106) and configured to receive the pair of bottom engagement protrusions (138B), and/or a nose engagement pocket (140C) disposed proximate a nose bridge portion (146) of the goggle frame (102) and configured to receive the nose engagement protrusion (138C).
A9.5. The goggle (100) of paragraph A9.4, wherein the pair of bottom engagement pockets (140B) each have an open lower end, such that respective bottom faces of the pair of bottom engagement protrusions (138B) are exposed to an external environment when the pair of bottom engagement protrusions (138B) are received in the pair of bottom engagement pockets (140B).
A9.6. The goggle (100) of paragraph A9.4 or A9.5, wherein the pair of bottom engagement protrusions (138B) are configured to fit into or under the pair of bottom engagement pockets (140B), such that the pair of bottom engagement pockets (140B) prevent the lens (112) from moving upward when the lens (112) is operatively received in the lens receiving area (106).
A9.7. The goggle (100) of any one of paragraphs A9.4-A9.6, wherein the top engagement pocket (140A) and the nose engagement pocket (140C) are configured to enclose the top engagement protrusion (138A) and the nose engagement protrusion (138C), respectively, when the top engagement protrusion (138A) and the nose engagement protrusion (138C) are operatively received in the top engagement pocket (140A) and the nose engagement pocket (140C), respectively.
A9.8. The goggle (100) of any one of paragraphs A9.1-A9.7, wherein engagement between the one or more engagement protrusions (138A-C) and the one or more engagement pockets (140A-C) is configured to prevent unwanted flexing of the goggle frame (102) during operation.
A9.9. The goggle (100) of any one of paragraphs A9.1-A9.8, wherein the one or more engagement protrusions (138A-C) and the one or more engagement pockets (140A-C) are configured to facilitate operative alignment of the lens (112) in the lens receiving area (106) during operation.
A9.10. The goggle (100) of any one of paragraphs A9.1-A9.9, wherein the one or more engagement pockets (140A-C) are formed in the first frame segment (104) and/or a second frame segment (148) of the goggle frame (102), wherein the second frame segment (148) is coupled to the first frame segment (104).
A9.11. The goggle (100) of paragraph A9.10, wherein a/the nose engagement pocket (140C) is formed in the second frame segment (148).
A10. The goggle (100) of any one of the preceding paragraphs, wherein the first frame segment (104) is configured to provide a structural support for the lens (112).
A11. The goggle (100) of any one of the preceding paragraphs, wherein the first frame segment (104) comprises:
a first canister (150A) (AKA first integral frame canister) configured to contain a supply roll (152) of a lens film (154); and
a second canister (150B) (AKA second integral frame canister) configured to contain a take-up roll (156) of the lens film (154);
wherein at least a respective portion of each of the first and second canisters (150A, 150B) is formed by an integral part (158A, 158B) of the first frame segment (104).
A11.1. The goggle (100) of paragraph A11, wherein the respective portion of each of the first and second canisters (150A, 150B) comprises a respective rear portion (160A, 160B) of the first and second canisters (150A, 150B).
A11.2. The goggle (100) of paragraph A11.1, wherein the respective rear portion (160A, 160B) comprises a respective spindle holder (162A, 162B) configured to removably retain the supply roll (152) or the take-up roll (156) during operation.
A11.3. The goggle (100) of any one of A11-A11.2, wherein the first (second) canister (150A, 150B) further comprises a first (second) cover (164A, 164B) configured to close the first (second) canister (150A, 150B) and contain the supply roll (152) or take-up roll (156) during operation.
A11.4. The goggle (100) of paragraph A11.3, wherein the first cover (164A) is configured to be selectively and operatively coupled to a first integral part (158A) of the first frame segment (104) to form the first canister (150A) and the second cover (164B) is configured to be selectively and operatively coupled to a second integral part (158B) of the first frame segment (104) to form the second canister (150B).
A11.5. The goggle (100) of paragraph A11.4, wherein the first cover (164A) comprises a first locking tab (166A) and the first integral part (158A) of the first frame segment (104) comprises a first tab opening (168A) configured to selectively and operatively receive the first locking tab (166A) to operatively couple the first cover (164A) to the first integral part (158A) of the first frame segment (104).
A11.6. The goggle (100) of any one of paragraphs A11.3-A11.5, wherein the second cover (164B) comprises a second locking tab (166B) and the second integral part (158B) of the first frame segment (104) comprises a second tab opening (168B) configured to selectively and operatively receive the second locking tab (166B) to operatively couple the second cover (164B) to the second integral part (158B) of the first frame segment (104).
A11.7. The goggle (100) of any one of paragraphs A11.3-A11.6, wherein the first and second covers (164A, 164B) are each configured to be removed and replaced repeatedly and manually without damage.
A11.8. The goggle (100) of any one of paragraphs A11-A11.8, wherein the first frame segment (104) and the integral parts (158A, 158B) of the first frame segment (104) that form the respective portions of the first and second canisters (150A, 150B) comprise or consist of a same material.
A11.9. The goggle (100) of any one of paragraphs A11-A11.8, wherein the first frame segment (104) and the first and second integral parts (158A, 158B) of the first frame segment (104) that form the respective portions of the first and second canisters (150A, 150B) are connected permanently by a single-use fastener.
A11.10. The goggle (100) of any one of paragraphs A11-A11.8, wherein the first frame segment (104) and the first and second integral parts (158A, 158B) of the first frame segment (104) that form the respective portions of the first and second canisters (150A, 150B) are molded as a single piece.
A11.11. The goggle (100) of any one of paragraphs A11-A11.10, wherein the first frame segment (104) and the first and second integral parts (158A, 158B) of the first frame segment (104) that form the respective portions of the first and second canisters (150A, 150B) comprise a continuous monolithic structure inseparable other than by using destructive methods.
A12. The goggle (100) of any one of the preceding paragraphs, wherein the goggle frame (102) further comprises a/the second frame segment (148) coupled to a rear of the first frame segment (104) (i.e., the side closer to the user's face when the goggle is worn), wherein the first and second frame segments comprise different materials.
A12.1. The goggle (100) of paragraph A12, wherein the first frame segment (104) is more rigid than the second frame segment (148).
A12.2. The goggle (100) of paragraph A12 or A12.1, wherein the second frame segment (148) is coupled to the first frame segment (104) by a plurality of dovetail joints (170).
A12.3. The goggle (100) of any one of paragraphs A12-12.2, wherein the second frame segment (148) comprises a lattice including a plurality of openings (137A-B).
A12.4. The goggle (100) of any one of paragraphs A12-A12.3, further comprising a foam portion (186) coupled to a rear side of the second frame segment (148), such that the foam portion (186) is configured to contact a user's face when the goggle (100) is worn.
A12.5. The goggle (100) of any one of paragraphs A12-A12.4, wherein one or more of a/the one or more engagement pockets (140A-C) are formed in the second frame segment (148).
A12.6. The goggle (100) of paragraph A12.5, wherein a/the nose engagement pocket (140C) is formed in the second frame segment (148).
A13. The goggle (100) of any one of the preceding paragraphs, wherein the lens (112) has a cylindrical contour or curvature.
A14. The goggle (100) of any one of the preceding paragraphs, wherein the lens (112) comprises an injection-molded plastic.
A15. The goggle (100) of any one of the preceding paragraphs, wherein the first lateral end of the lens (112) is configured to be pivotably coupled to the first frame segment (104) by the hook (114) operatively engaged with the pin (108) and the second lateral end of the lens (112) is configured to be releasably latched to the first frame segment (104) by the latch locking member (110) operatively mated with the latch-receiving aperture (116).
A16. The goggle (100) of any one of the preceding paragraphs, wherein the lens (112) comprises a/the lower nose cutout (172).
A17. The goggle (100) of any one of the preceding paragraphs, wherein an upper/top edge of the lens (112) comprises a raised central portion (174).
A17.1. The goggle (100) of paragraph A17, wherein the raised central portion (174) extends upward from lateral portions of the upper/top edge of the lens (112).
A18. The goggle (100) of any one of the preceding paragraphs, further comprising a head strap (176) coupled to the goggle frame (102).
A18.1. The goggle (100) of paragraph A18, wherein the head strap (176) is operatively coupled to the first frame segment (104) of the goggle frame (102).
A18.2. The goggle (100) of paragraph A18 or A18.1, wherein a first end of the head strap (176) is plugged into a/the first integral part (158A) of the first frame segment (104) forming a/the first rear portion (160A) of a/the first canister (150A) and a second end of the head strap (176) is plugged into a/the second integral part (158B) of the first frame segment (104) forming a/the second rear portion (160B) of a/the second canister (150B).
A18.3. The goggle (100) of paragraph A18.2, wherein the first end of the head strap (176) is plugged into a first vertical recess (178A) formed in the first rear portion (160A) of the first canister (150A) and the second end of the head strap (176) is plugged into a second vertical recess (178B) formed in the second rear portion (160B) of the second canister (150B).
A18.4. The goggle (100) of any one of paragraphs A18-A18.3, wherein the head strap (176) comprises a length-adjustable strip of material.
A18.5. The goggle (100) of any one of paragraphs A18-A18.4, wherein the material is elastic.
A19. The goggle (100) of any one of the preceding paragraphs, further comprising a/the supply roll (152) of lens film (154) and a/the take-up roll (156).
A19.1. The goggle (100) of paragraph A19, wherein the lens film (154) extends across the lens (112) and is coupled to the take-up roll (156).
A19.2. The goggle (100) of paragraph A19 or A19.1, further comprising a pull-cord ratchet mechanism (180) configured to index the lens film (154) across the lens (112).
A19.3. The goggle (100) of paragraph A19.2, wherein the take-up roll (156) is operatively mounted on a take-up spindle (151B) of the second canister (150B), and wherein the pull-cord ratchet mechanism (180) is operatively coupled to the take-up spindle (151B), such that actuation of the pull-cord ratchet mechanism (180) rotates the take-up spindle (151B) and the take-up roll (156) mounted on the take-up spindle (151B) to cause the lens film (154) to be indexed across the lens (112).
A20. The goggle (100) of any one of the preceding paragraphs, further comprising a mud visor (182) configured to be operatively and releasably coupled to the goggle frame (102).
A20.1. The goggle (100) of paragraph A20, wherein the mud visor (182) is configured to cover and extend over an upper surface of the first frame segment (104) and a/the first and second canisters (150A, 150B) when the mud visor (182) is operatively coupled to the goggle frame (102).
A20.2. The goggle (100) of paragraph A20 or A20.1, wherein the mud visor (182) comprises a mud flap (184) configured to extend in front of an upper portion of the lens (112) when the mud visor (182) is operatively coupled to the goggle frame (102).
A20.3. The goggle (100) of paragraph A20.2, wherein the mud flap (184) is configured to extend in front of a/the lens film (154) extending across the lens (112) when the mud visor (182) is operatively coupled to the goggle frame (102).
A20.4. The goggle (100) of any one of paragraphs A20-A20.3, wherein the mud visor (182) is configured to prevent dirt and debris from entering between the lens film (154) and the lens (112) at an upper edge of the lens film (154).
A20.5. The goggle (100) of any one of paragraphs A20-A20.4, wherein the mud visor (182) comprises a flexible/elastic material configured to deform to permit selectively coupling and decoupling of the mud visor (182) to the goggle frame (102).
A20.6. The goggle (100) of paragraph A20.5, wherein the mud visor (182) is configured to operatively couple to the goggle frame (102) in a press fit arrangement.
A21. The goggle (100) of any one of the preceding paragraphs, wherein the lens (112) is configured to be operatively coupled to the goggle frame (102) by operatively engaging the hook (114) with the pin (108), pivoting the lens (112) about the pin (108), such that the second lateral end of the lens (112) is received in the lens receiving area (106), and securing the lens (112) in the lens receiving area (106) by operatively mating the latch locking member (110) with the latch-receiving aperture (116).
A22. The goggle (100) of any one of the preceding paragraphs, wherein the lens (112) is configured to abut a front surface of the first frame segment (104) when the lens (112) is operatively received in the lens receiving area (106).
B0. A protective goggle for motorsports, the goggle comprising:
a goggle frame (102) having a first frame segment (104).
C0. The goggle (100) of paragraph B0, wherein the first frame segment (104) comprises:
a first canister (150A) (AKA first integral frame canister) configured to contain a supply roll (152) of a lens film (154); and
a second canister (150B) (AKA second integral frame canister) configured to contain a take-up roll (156) of the lens film (154);
wherein at least a respective portion of each of the first and second canisters (150A, 150B) comprises an integral part (158A, 158B) of the first frame segment (104).
C1. The goggle (100) of paragraph C0, wherein the respective portion of each of the first and second canisters (150A, 150B) comprises a respective rear portion (160A, 160B) of the first and second canisters (150A, 150B).
C2. The goggle (100) of paragraph C1, wherein the respective rear portion (160A, 160B) comprises a respective spindle holder (162A, 162B) configured to removably retain the supply roll (152) or the take-up roll (156) during operation.
C3. The goggle (100) of any one of paragraphs C0-C2, wherein the first (second) canister (150A, 150B) further comprises a first (second) cover (164A, 164B) configured to close the first (second) canister 150A, 150B) and contain the supply roll (152) or take-up roll (156) during operation.
C3.1. The goggle (100) of paragraph C3, wherein the first cover (164A) is configured to be selectively and operatively coupled to a first integral part (158A) of the first frame segment (104) to form the first canister (150A) and the second cover (164B) is configured to be selectively and operatively coupled to a second integral part (158B) of the first frame segment (104) to form the second canister (150B).
C3.2. The goggle (100) of paragraph C3 or 3.1, wherein the first cover (164A) comprises a first locking tab (166A) and the first integral part (158A) of the first frame segment (104) comprises a first tab opening (168A) configured to selectively and operatively receive the first locking tab (166A) to operatively couple the first cover (164A) to the first integral part (158A) of the first frame segment (104).
C3.3. The goggle (100) of any one of paragraphs C3-C3.2, wherein the second cover (164B) comprises a second locking tab (166B) and the second integral part (158B) of the first frame segment (104) comprises a second tab opening (168B) configured to selectively and operatively receive the second locking tab (166B) to operatively couple the second cover (164B) to the second integral part (158B) of the first frame segment (104).
C3.4. The goggle (100) of any one of paragraphs C3-C3.3, wherein the first and second covers (164A, 164B) are each configured to be removed and replaced repeatedly and manually without damage.
C4. The goggle (100) of any one of paragraphs C0-C3.4, wherein the first frame segment (104) and the integral parts (158A, 158B) of the first frame segment (104) that form the respective portions of the first and second canisters (150A, 150B) comprise or consist of a same material.
C5. The goggle (100) of any one of paragraphs C0-C4, wherein the first frame segment (104) and the integral parts (158A, 158B) of the first frame segment (104) that form the respective portions of the first and second canisters (150A, 150B) are connected permanently by a single-use fastener.
C6. The goggle (100) of any one of paragraphs C0-C4, wherein the first frame segment (104) and the integral parts (158A, 158B) of the first frame segment (104) that form the respective portions of the first and second canisters (150A, 150B) are molded as a single piece.
C7. The goggle (100) of any one of paragraphs C0-C6, wherein the first frame segment (104) and the integral parts (158A, 158B) of the first frame segment (104) that form the respective portions of the first and second canisters (150A, 150B) comprise a continuous monolithic structure inseparable other than by using destructive methods.
C8. The goggle (100) of any one of paragraphs C0 through C7, wherein the goggle frame (102) further comprises a second frame segment (148) coupled to a rear of the first frame segment (104) (i.e., the side closer to the user's face when worn), wherein the first and second frame segments comprise different materials.
C8.1. The goggle (100) of C8, wherein the first frame segment (104) is more rigid than the second frame segment (148).
C8.2. The goggle (100) of C8 or C8.1, wherein the second frame segment (148) is coupled to the first frame segment (104) by a plurality of dovetail joints (170).
C8.3. The goggle (100) of any one of C8-C8.2, wherein the second frame segment (148) (100) comprises a lattice including a plurality of openings.
C8.4. The goggle (100) of any one of C8-C8.3, further comprising a foam portion coupled to a rear side of the second frame segment (148), such that the foam portion is configured to contact a user's face when the goggle (100) is worn.
C9. The goggle (100) of any one of C0 through C8.4, further comprising the lens (112), the lens (112) having a first lateral end and a second lateral end.
C9.1. The goggle (100) of C9, wherein the lens (112) has a cylindrical contour or curvature.
C9.2. The goggle (100) of C9 or C9.1, wherein the lens (112) comprises an injection-molded plastic.
C9.3. The goggle (100) of any one of C9 through C9.2, wherein the first lateral end of the lens (112) comprises an integral hook (114).
C9.4. The goggle (100) of C9.3, wherein the first frame segment (104) comprises a pin (108) configured to mate with the hook (114) of the first lateral end of the lens (112).
C9.5. The goggle (100) of C9.4, wherein the lens (112) is pivotable about the pin (108) when the hook (114) is engaged with the pin (108).
C9.6. The goggle (100) of any one of C9 through C9.5, wherein the second lateral end of the lens (112) comprises a latch-receiving aperture (116).
C9.7. The goggle (100) of C9.6, wherein the latch-receiving aperture (116) is formed in a forward projecting tab (134) of the lens (112).
C9.8. The goggle (100) of C9.6 or C9.7, wherein the goggle frame (102) further comprises a latch locking member (110) configured to releasably mate with the latch-receiving aperture (116) of the lens (112) to selectively retain the second lateral end of the lens (112) on the goggle frame (102).
C9.9 The goggle (100) of C9.8, wherein an end of the latch locking member (110) is accessible on a rear side of the first frame segment (104), and the latch locking member (110) is configured such that manipulation of the end of the latch locking member (110) causes selective insertion and retraction of the latch locking member (110) with respect to the latch-receiving aperture (116).
C9.10 The goggle (100) of any one of C9 through C9.9, wherein the first lateral end of the lens (112) is configured to be pivotably coupled to the first frame segment (104) and the second lateral end of the lens (112) is configured to be releasably latched to the first frame segment (104).
C9.11. The goggle (100) of any one of C9 through C9.10, wherein a lower peripheral edge of the lens (112) comprises a plurality of rearward projections (138A-C).
C9.12. The goggle (100) of C9.11, wherein the plurality of rearward projections (138A-C) are configured to fit into or under corresponding pockets or gaps (140A-C) of the goggle frame (102), such that the rearward projections (138A-C) prevent the lens (112) from moving upward when the lens (112) is mounted to the goggle frame (102).
C9.13 The goggle (100) of any one of C9-C9.12, wherein the lens (112) comprises a lower nose cutout (144).
C9.14. The goggle (100) of any one of C9-C9.13, wherein an upper edge of the lens (112) comprises a raised central portion (174).
C10. The goggle (100) of any one of C0 through C9.14, further comprising a head strap (176) coupled to the first (second) canister (150A, 150B).
C10.1. The goggle (100) of C10, wherein the head strap (176) comprises a length-adjustable strip of material.
C10.2. The goggle (100) of C10.1, wherein the material is elastic.
C10.3. The goggle (100) of any one of C10 through C10.2, wherein a first end of the head strap (176) is plugged into a vertical recess (178A, 178B) in a rear surface of the first (second) canister (150A, 150B).
C10.4. The goggle (100) of C10.3, wherein a second end of the head strap (176) is plugged into the second (first) canister (150A, 150B).
C11. The goggle (100) of any one of C0 through C10.4, further comprising the supply roll (152) of lens film (154).
C11.1. The goggle (100) of C11, further comprising the take-up roll (156), wherein the lens film (154) extends across the lens (112) and is coupled to the take-up roll.
C11.2. The goggle (100) of C11 or C11.1, further comprising a pull-cord ratchet mechanism (180) configured to index the lens film (154) across the lens (112).
C12. The goggle (100) of any one of C0 through C11.2, wherein the first frame segment (104) forms a peripheral rim (105) that extends around a complete periphery or forms a closed loop or defines a fully enclosed lens receiving area (106).
C13. The goggle (100) of any one of paragraphs C0-C12, further comprising a mud visor (182) configured to be operatively and releasably coupled to the goggle frame (102).
C13.1. The goggle (100) of paragraph C13, wherein the mud visor (182) is configured to cover and extend over the first frame segment (104) and a/the first and second canisters (150A, 150B) when the mud visor (182) is operatively coupled to the goggle frame (102).
C13.2. The goggle (100) of paragraph C13 or C13.1, wherein the mud visor (182) comprises a mud flap (184) configured to extend in front of an upper portion of the lens (112) when the mud visor (182) is operatively coupled to the goggle frame (102) and the lens (112) is operatively coupled to the goggle frame (102).
C13.3. The goggle (100) of paragraph C13.2, wherein the mud flap (184) is configured to extend in front of a/the lens film (154) extending across the lens (112) when the mud visor (182) is operatively coupled to the goggle frame (102).
C13.4. The goggle (100) of any one of paragraphs C13-C13.3, wherein the mud visor (182) is configured to prevent dirt and debris from entering between the lens film (154) and the lens (112) at an upper edge of the lens film (154).
C13.5. The goggle (100) of any one of paragraphs C13-C13.4, wherein the mud visor (182) comprises a flexible/elastic material configured to be selectively deformable to permit selectively releasably coupling the mud visor (182) to the goggle frame (102).
C13.6. The goggle (100) of paragraph C13.5, wherein the mud visor (182) is configured to operatively couple to the goggle frame (102) in a press fit arrangement.
D0. The goggle (100) of B0, further comprising:
a lens (112) having a first lateral end pivotably coupled to a pin (108) of the first frame segment (104) and a second lateral end releasably captured by a latch (111) of the first frame segment (104).
D1. The goggle (100) of D0, wherein the lens (112) has a cylindrical contour or curvature.
D2. The goggle (100) of D0 or D1, wherein the lens (112) comprises an injection-molded plastic.
D3. The goggle (100) of any one of D0 through D2, wherein the first lateral end of the lens (112) comprises an integral hook (114).
D4. The goggle (100) of D3, wherein the pin (108) of the first frame segment (104) is configured to mate with the integral hook (114) of the first lateral end of the lens (112).
D5. The goggle (100) of D4, wherein the lens (112) is pivotable about the pin (108) when the integral hook (114) is engaged with the pin (108).
D6. The goggle (100) of any one of D0 through D5, wherein the second lateral end of the lens (112) comprises a latch-receiving aperture (116).
D7. The goggle (100) of D6, wherein the latch-receiving aperture (116) is formed in a forward projecting tab (134) of the lens (112).
D8. The goggle (100) of D6 or D7, wherein a latch locking member (110) of the goggle frame (102) is configured to releasably mate with the latch-receiving aperture (116) of the lens (112) to selectively retain the first lateral end of the lens (112) on the goggle frame (102).
D9. The goggle (100) of D8, wherein an end of the latch locking member (110) is accessible on a rear side of the first frame segment (104), and the latch locking member (110) is configured such that manipulation of the end of the latch locking member (110) causes selective insertion and retraction of the latch locking member (110) with respect to the latch-receiving aperture (116).
D10. The goggle (100) of D9, further comprising an abutment configured to limit movement of the latch locking member (110).
D11. The goggle (100) of any one of D0 through D10, wherein a lower peripheral edge of the lens (112) comprises a plurality of rearward projections (138A-C).
D12. The goggle (100) of D11, wherein the plurality of rearward projections (138A-C) are configured to fit into or under corresponding pockets or gaps (140A-C) of the goggle frame (102), such that the rearward projections (138A-C) prevent the lens (112) from moving upward when the lens (112) is mounted to the goggle frame (102).
D13. The goggle (100) of any one of D0 through D12, further comprising one or more rearward projections (138A) extending rearward from an upper peripheral edge of the lens (112) configured to fit into a corresponding engagement pocket (140A) of the goggle frame.
D14. The goggle (100) of any one of D0 through D13, wherein the lens (112) comprises a lower nose cutout (144).
D15. The goggle (100) of any one of D0 through D14, wherein an upper edge of the lens (112) comprises a raised central portion (174).
D16. The goggle (100) of any one of D0 through D15, wherein the first frame segment (104) forms a peripheral rim (105) that extends around a complete periphery or forms a closed loop or defines a fully enclosed lens area (106).
D17. The goggle (100) of any one of D0 through D16, further comprising the first and second integral frame canisters (150A, 150B) of any one of paragraphs C0 through C18.
E0. A protective goggle (100), comprising:
a goggle frame (102) including a lens receiving area (106), wherein the goggle frame (102) includes a pin (108) disposed at a first lateral end of the lens receiving area (106) and a latch mechanism (111) disposed at a second lateral end of the lens receiving area (106); and
a lens (112) configured to be operatively received in the lens receiving area (106), wherein a first lateral end of the lens (112) is configured to be pivotably coupled to the pin 108 and a second lateral end of the lens (112) is configured to be latched to the goggle frame (102) by the latch mechanism (111) to secure the lens (112) in the lens receiving area (106).
E1. The goggle of paragraph E0, further comprising the features, structures, and/or components of any one of paragraphs A0-A22.
F0. A protective goggle (100), comprising:
a goggle frame (102) including a lens receiving area (106); and
a lens (112) configured to be operatively received in the lens receiving area (106), wherein a first end of the lens (112) is configured to be pivotably coupled to the goggle frame (102) and a second end of the lens (112) is configured to be latched to the goggle frame (102) to secure the lens (112) in the lens receiving area (106).
F1. The goggle of paragraph F0, further comprising the features, structures, and/or components of any one of paragraphs A0-A22.
G0. A method (200) of assembling a protective goggle (100), the method comprising:
pivotably coupling a first lateral end of a lens to a goggle frame;
pivoting the lens relative to the goggle frame, such that a second lateral end of the lens is received in a lens receiving area of the goggle frame; and
latching the second lateral end of the lens to the goggle frame to secure the lens in the lens receiving area.
G1. The method (200) of paragraph G0, wherein the lens comprises a hook disposed at the first lateral end of the lens and the frame includes a pin disposed at a first lateral end of the lens receiving area, wherein pivotably coupling the first lateral end of the lens to the goggle frame includes operatively engaging the hook with the pin.
G1.1. The method (200) of paragraph G1, wherein the pivoting the lens relative to the goggle frame comprises pivoting the lens about the pin with the hook operatively engaged with the pin.
G2. The method (200) of any one of paragraphs G0-G1.1, wherein the goggle frame comprises a latch locking member, wherein latching the second lateral end of the lens to the goggle frame includes transitioning the latch locking member into a locked position, in which the latch locking member operatively engages the second lateral end of the lens.
G2.1. The method (200) of paragraph G2, wherein the second lateral end of the lens comprises a latch-receiving aperture, wherein the latch locking member is operatively mated with the latch-receiving aperture when in the locked position.
G2.2. The method (200) of paragraph G2, wherein the lens includes an integral tab projecting forward from a front surface of the lens, wherein the latch-receiving aperture is formed in the integral tab.
G3. The method (200) of any one of paragraphs G0-G2.2, further comprising indexing a lens film across the lens from a supply roll of the lens film to a take-up roll.
G3.1. The method (200) of paragraph G3, wherein the supply roll is disposed in a first integral canister of the goggle frame and the take-up roll is disposed in a second integral canister of the goggle frame.
G3.2. The method (200) of paragraph G3 or G3.1, wherein the first and second integral canisters are disposed on opposing lateral sides of the lens receiving area.
G3.3. The method (200) of any one of paragraphs G3.1-G3.2, further comprising releasably coupling first and second canister covers 164A, 164B to the goggle frame 102 to enclose the supply roll in the first integral canister and enclose the take-up roll in the second integral canister.
G4. The method (200) of any one of paragraphs G0-G2.2, further comprising releasably coupling a mud visor to an upper portion of the goggle frame, such that a mud flap of the mud visor extends downward in front of an upper portion of the lens.
G4.1. The method (200) of paragraph G4, wherein the mud flap extends in front of and covers an upper edge of the lens film to prevent dirt and/or debris from entering between the lens and the lens film at the upper edge of the lens film.
H0. A protective goggle, comprising:
a goggle frame including a first frame segment forming a lens receiving area configured to receive a lens of the goggle, wherein the goggle frame further comprises:
a first integral canister configured to contain a supply roll of a lens film; and
a second integral canister configured to contain a take-up roll of the lens film;
wherein a first integral part of the goggle frame forms at least a first portion of the first integral canister and a second integral part of the goggle frame forms at least a second portion of the second integral canister.
H1. The goggle of paragraph H0, wherein the first and second integral parts form rear portions of the first and second integral canisters, respectively.
H2. The goggle of paragraph H0 or H1, wherein the first and second integral parts form forward portions of the first and second integral canisters, respectively.
H3. The goggle of any one of paragraphs H0-H2, wherein the first and second integral canisters each comprise a respective spindle holder configured to removably retain the supply roll or the take-up roll during operation.
H4. The goggle of any one of paragraphs H0-H3, wherein the first and second integral parts are integral parts of the first frame segment.
H5. The goggle of any one of paragraphs H0-H3, wherein the goggle frame further comprises a second frame segment operatively coupled to a rear side of the first frame segment.
H5.1. The goggle of paragraph H5, wherein the first and second integral parts are integral parts of the second frame segment.
H5.2. The goggle of paragraph H5 or H5.1, wherein the first frame segment comprises a first material and the second frame segment comprises a second material different than the first material.
H5.3. The goggle of paragraph H5.2, wherein the first material has greater rigidity than the second material.
H5.4. The goggle of any one of paragraphs H5-H5.3, further comprising a face contacting layer operatively coupled to a rear side of the second frame segment and configured to contact a user's face when the goggle is worn.
H6. The goggle of any one of paragraphs H0-H5.4, wherein the first integral canister includes a first canister cover configured to be selectively and operatively coupled to the first integral part to enclose the supply roll within the first integral canister, and wherein the second integral canister includes a second canister cover configured to be selectively and operatively coupled to the second integral part to enclose the take-up roll within the second integral canister.
H6.1. The goggle of paragraph H6, wherein the first canister cover is configured to cover a first lateral side of the lens receiving area and the second canister cover is configured to cover a second lateral side of the lens receiving area when the first and second canister covers are operatively coupled to the first and second integral parts.
H6.2. The goggle of paragraph H6 or H6.1, wherein the first canister cover forms a first front portion of the first integral canister when operatively coupled to the first integral part and the second canister cover forms a second front portion of the second integral canister when operatively coupled to the second integral part.
H7. The goggle of any one of paragraphs H0-H6.2, further comprising a mud visor configured to be selectively and operatively coupled to the goggle frame, wherein the mud visor is configured to cover an upper surface of the first frame segment and the first and second integral canisters when the mud visor is operatively coupled to the goggle frame.
H7.1. The goggle of paragraph H7, wherein the mud visor comprises an elastic material configured to elastically deform to permit selectively coupling and decoupling the mud visor to the goggle frame.
H7.2. The goggle of paragraph H7.1, wherein the first and second integral canisters include first and second canister covers configured to be selectively and operatively coupled to the first and second integral parts to enclose the supply roll or the take-up roll within the first and second integral canisters, and wherein the mud visor is configured to apply an elastic force on the first and second canister covers to hold the first and second canister covers on the first and second integral parts.
H8. The goggle of any one of paragraphs H0-H7.2, further comprising the lens configured to be operatively coupled to the first frame segment, wherein the lens includes a first lateral end and a second lateral end.
H8.1. The goggle of paragraph H8, further comprising a first latch mechanism operatively supported by the first integral part and a second latch mechanism operatively supported by the second integral part, wherein the first and second latch mechanisms are configured to selectively and operatively engage the first and second lateral ends of the lens to secure the lens to the goggle frame.
H8.2. The goggle of paragraph H8.1, wherein the lens includes a first latch-engagement tab disposed at the first lateral end and a second latch-engagement tab disposed at the second lateral end, wherein the first latch mechanism is configured to selectively and operatively engage the first latch-engagement tab and the second latch mechanism is configured to selectively and operatively engage the second latch-engagement tab.
H8.3. The goggle of paragraph H8.1 or H8.2, wherein the first latch mechanism is slidingly coupled to a first rear side of the first integral part and the second latch mechanism is slidingly coupled to a second rear side of the second integral part.

### Advantages, Features, and Benefits

The different embodiments and examples of the protective sports goggles described herein provide several advantages over known sports goggles. For example, illustrative embodiments and examples described herein allow a secure and selectively releasable connection between the lens and the goggle frame, which permits ease of assembly and disassembly for the user, while preventing unwanted movement or disconnection of the lens relative to the goggle frame during use.

Additionally, and among other benefits, illustrative embodiments and examples described herein allow a single-piece first frame segment that forms a lens receiving area configured to receive a lens of the goggle, at least a portion of a pair of film canisters disposed on opposing sides of the lens receiving area, and a head strap of the goggle may be coupled to the same single-piece first frame segment. The single-piece first frame segment of the goggles described herein increases the strength and rigidity of the goggle, reduces manufacturing costs and complexity, and/or may improve the aesthetic appearance of the goggle in comparison to designs having multiple connected frame portions that perform different functions.

Additionally, and among other benefits, illustrative embodiments and examples described herein allow a film dispensing system integrated into a goggle frame of the goggle that permits the dispensing of protective lens film to cover the front surface of the lens during use.

No known system or device can perform these functions. However, not all embodiments and examples described herein provide the same advantages or the same degree of advantage.

### CONCLUSION

The disclosure set forth above may encompass multiple distinct examples with independent utility. Although each of these has been disclosed in its preferred form(s), the specific embodiments thereof as disclosed and illustrated herein are not to be considered in a limiting sense, because numerous variations are possible. To the extent that section headings are used within this disclosure, such headings are for organizational purposes only. The subject matter of the disclosure includes all novel and nonobvious combinations and subcombinations of the various elements, features, functions, and/or properties disclosed herein. The following claims particularly point out certain combinations and subcombinations regarded as novel and nonobvious. Other combinations and subcombinations of features, functions, elements, and/or properties may be claimed in applications claiming priority from this or a related application. Such claims, whether broader, narrower, equal, or different in scope to the original claims, also are regarded as included within the subject matter of the present disclosure.

1. A protective goggle, comprising:
a goggle frame including a first frame segment forming a lens receiving area configured to receive a lens of the goggle, wherein the goggle frame further comprises:
a first integral canister configured to contain a supply roll of a lens film; and
a second integral canister configured to contain a take-up roll of the lens film;
wherein a first integral part of the goggle frame forms at least a first portion of the first integral canister and a second integral part of the goggle frame forms at least a second portion of the second integral canister.

2. The goggle of claim 1, wherein the first and second integral parts form rear portions of the first and second integral canisters, respectively.

3. The goggle of claim 1, wherein the first and second integral parts form forward portions of the first and second integral canisters, respectively.

4. The goggle of claim 1, wherein the first and second integral canisters each comprise a respective spindle holder configured to removably retain the supply roll or the take-up roll during operation.

5. The goggle of claim 1, wherein the first and second integral parts are integral parts of the first frame segment.

6. The goggle of claim 1, wherein the goggle frame further comprises a second frame segment operatively coupled to a rear side of the first frame segment.

7. The goggle of claim 6, wherein the first and second integral parts are integral parts of the second frame segment.

8. The goggle of claim 6, wherein the first frame segment comprises a first material and the second frame segment comprises a second material different than the first material.

9. The goggle of claim 8, wherein the first material has greater rigidity than the second material.

10. The goggle of claim 6, further comprising a face contacting layer operatively coupled to a rear side of the second frame segment and configured to contact a user's face when the goggle is worn.

11. The goggle of claim 1, wherein the first integral canister includes a first canister cover configured to be selectively and operatively coupled to the first integral part to enclose the supply roll within the first integral canister, and wherein the second integral canister includes a second canister cover configured to be selectively and operatively coupled to the second integral part to enclose the take-up roll within the second integral canister.

12. The goggle of claim 11, wherein the first canister cover is configured to cover a first lateral side of the lens receiving area and the second canister cover is configured to cover a second lateral side of the lens receiving area when the first and second canister covers are operatively coupled to the first and second integral parts.

13. The goggle of claim 11, wherein the first canister cover forms a first front portion of the first integral canister when operatively coupled to the first integral part and the second canister cover forms a second front portion of the second integral canister when operatively coupled to the second integral part.

14. The goggle of claim 1, further comprising a mud visor configured to be selectively and operatively coupled to the goggle frame, wherein the mud visor is configured to cover an upper surface of the first frame segment and the first and second integral canisters when the mud visor is operatively coupled to the goggle frame.

15. The goggle of claim 14, wherein the mud visor comprises an elastic material configured to elastically deform to permit selectively coupling and decoupling the mud visor to the goggle frame.

16. The goggle of claim 15, wherein the first and second integral canisters include first and second canister covers configured to be selectively and operatively coupled to the first and second integral parts to enclose the supply roll or the take-up roll within the first and second integral canisters, and wherein the mud visor is configured to apply an elastic force on the first and second canister covers to hold the first and second canister covers on the first and second integral parts.

17. The goggle of claim 1, further comprising the lens configured to be operatively coupled to the first frame segment, wherein the lens includes a first lateral end and a second lateral end.

18. The goggle of claim 17, further comprising a first latch mechanism operatively supported by the first integral part and a second latch mechanism operatively supported by the second integral part, wherein the first and second latch mechanisms are configured to selectively and operatively engage the first and second lateral ends of the lens to secure the lens to the goggle frame.

19. The goggle of claim 18, wherein the lens includes a first latch-engagement tab disposed at the first lateral end and a second latch-engagement tab disposed at the second lateral end, wherein the first latch mechanism is configured to selectively and operatively engage the first latch-engagement tab and the second latch mechanism is configured to selectively and operatively engage the second latch-engagement tab.

20. The goggle of claim 18, wherein the first latch mechanism is slidingly coupled to a first rear side of the first integral part and the second latch mechanism is slidingly coupled to a second rear side of the second integral part.
